(19) United States
(12) Patent Application Publication
(10) Pub. No.: US 2007/0020711 A1
(43) Pub. Date: Jan. 25, 2007

(54) Fungal antigen immunoassay

(76) Inventor: L. Joseph Wheat, Indianapolis, IN (US)

Correspondence Address: INDIANAPOLIS OFFICE 27879;BRINKS HOFER GILSON & LIONE, ONE INDIANA SQUARE, SUITE 1600, INDIANAPOLIS, IN, 46204-2033, US

(21) Appl. No.: 11/491,647

(22) Filed: Jul. 24, 2006

Related U.S. Application Data
(60) Provisional application No. 60/702,653, filed on Jul. 25, 2005.

(51) Int. Cl. G01N 33/569; G01N 33/53

(52) U.S. Cl. 435/7.31

(57) ABSTRACT

The present disclosure relates to methods for detecting a fungal antigen in a physiological specimen. The present invention includes methods and materials for testing for antigens associated with endemic mycoses as well as quantitative analysis of the test results.

### RELATED APPLICATION

This application claims the benefit of U.S. provisional patent application Ser. No. 60/702,653, entitled “FUNGAL ANTIGEN IMMUNOASSAY,” filed Jul. 25, 2005 and incorporated herein by reference in its entirety.

### FIELD OF THE INVENTION

The present invention relates to the field of medical diagnostics, particularly with respect to fungal pathogens. More particularly, the present invention relates to immunoassay detection of fungal antigens, including Histoplasma capsulatum.

### BACKGROUND OF THE INVENTION

Histoplasmosis is acquired by the inhalation of the mold form of Histoplasma capsulatum, known as microconidia, which transforms to the yeast form in tissues. H. capsulatum is a pathogenic dimorphic fungus that grows as multicellular mycelia in nature, and as unicellular budding yeasts in humans and animals. Inhalation of airborne propagules results in a morphological transformation to the yeast form which may cause pulmonary infection and occasional progressive disease, particularly in immunosuppressed patients. Histoplasmosis is believed to be highly endemic in the Ohio and Mississippi valley regions of the United States. Most H. capsulatum infections are not clinically recognized, but are identified as incidental radiographic or pathological findings. Furthermore, most symptomatic cases of otherwise healthy individuals are mild and are resolved without therapy. Among healthy individuals who are symptomatic, most present with acute pulmonary histoplasmosis that appears flu-like. Pericarditis or rheumatological syndromes are less common manifestations of acute histoplasmosis in otherwise healthy patients. Patients with underlying diseases may develop progressive histoplasmosis, which is a chronic pulmonary infection in those with emphysema and progressive disseminated disease (i.e., spreading systemically to other organs of the body) in those with AIDS or other diseases associated with immunosuppression. See Wheat and Kauffman, Infect. Dis. Clin. North Am. 17:1-19, vii (2003).

The diagnosis of histoplasmosis in humans is often suggested by results of a careful clinical evaluation and radiologic studies, but laboratory tests are necessary to confirm the diagnosis. Isolation of the organism from blood or tissue provides a definitive diagnosis. Serological tests are also an important diagnostic tool for histoplasmosis. The most widely available tests are the immunodiffusion assay, which detects antibodies to heat-sensitive glycoproteins called H and M antigens, and the more sensitive complement fixation test, which is traditionally performed with yeast and mycelial antigens. More sensitive antibody assays such as radioimmunoassay and enzyme immunoassay have been used to detect IgM and IgG antibodies to fungal extracts. Enzyme linked immunoabsorbant assay (ELISA) is a sensitive analytical technique used for determination of the concentration of certain antigens and antibodies. ELISA is a useful tool in disease diagnosis, including detection of fungal infection such as Histoplasma. ELISA is typically performed using a polystyrene microtiter detection plate with a capture antibody or antigen immobilized onto the surface of the wells of the microtiter plate.

ELISA testing can be used for the diagnosis of histoplasmosis. FIG. 1 is a schematic of an enzyme-linked immunoassay (ELISA) system 10 showing a positive detection configuration in the presence of an antigen and a.detection antibody in a sandwich enzyme immunoassay. The immunoassay system 10 can detect the presence of an antigen 40 in an analyte, such as serum or urine by contacting the analyte with an antigen binding surface. The antigen binding surface is typically a well in a detection plate having a capture antibody 20 attached to the surface 12 of a detection plate well. A non-specific blocking reagent 30 can be contacted with the detection plate well in a manner effective to bind the blocking reagent 30 to the surface 12 of the detection plate well without displacing the capture antibody 20. Once prepared, the antigen binding surface is contacted with an analyte comprising the antigen 40 in a manner effective to bind the antigen 40 to the capture antibody 20. A detection antibody 50 can be contacted with the surface 12 after the analyte solution in a manner effective to bind the detection antibody 50 to the antigen 40 attached to the capture antibody 20. Preferably, the analyte solution is removed from the surface 12 prior to contacting the detection antibody 50 with the surface 12. The detection antibody 50 can include an antibody portion and a detection portion, which can include a reporting element. The antibody portion is configured to bind to a desired antigen 40, while the detection portion is adapted to permit detection by a suitable method, such as a color change. The presence of the antigen 40 is identified by detecting the presence of the detection antibody 50. ELISA protocols can be designed in a heterogeneous format or a homogeneous format. A standard ELISA using a heterogeneous format involves a series of incubations of a surface with a reagent contained in a physiological buffer separated by washes to remove material that did not bind to the surface. In contrast, a homogeneous format ELISA includes no requirement for wash steps between incubations of the surface with the various reagents, such as is the case with the commercially-available CEDIA® (Boehringer Mannheim Gmbh) and EMIT® (Behring Diagnostics Inc.) technologies that are currently in use with other immunoassays. The enzyme-linked immunoassay system 10 can be configured to detect one or more antigen in an analyte sample.

However, the reliability of these tests can be hampered by false positive or false negative reactions, particularly in individuals unknowingly carrying human anti-animal antibodies. See, e.g., L. J. Kricka, “Human Anti-Animal Antibody Interferences in Immunological Assays,” Clinical Chemistry 45:7, 942-956 (1999). The efficacy of such an ELISA antigen detection system can be compromised by molecules that interfere with binding between the capture antibody and the antigen, or interfere with antigen binding to the detector antibody. The former can lead to false positive indications for the antigen, the latter to false negative indications. FIG. 2A is a schematic of a false-positive reading in the enzyme-linked immunoassay system 10 shown in FIG. 1, whereby a human anti-animal antibody 140 binds to the capture antibody 120 and a detection antibody 150 binds to the human anti-animal antibody 140. The detection signal from bound detection antibody 150 can be mis-attributed to the binding of the antigen in FIG. 1, leading to a false positive reading. FIG. 2B is a schematic of a false-negative reading in the enzyme immunoassay system 200, similar to the system shown in FIGS. 1 and 2A. A first human anti-animal antibody 240 binds to the capture antibody 220 in the presence of a non-specific blocking agent 230 bound to the surface 212, blocking the antigen binding site. A second human anti-animal antibody 242 binds to a detector antibody 250 in solution, preventing the capture antibody 250 from binding to a capture antibody 220.

Human anti-animal antibodies typically go undetected in patients, often resulting in false positive or false negative readings from ELISA tests for pathogenic antigens, such as Histoplasma capsulatum. False positive readings can result in unnecessary medical intervention, while false negative readings can lead to mis-diagnosis or failure to administer appropriate medical care. Human anti-animal antibodies are more likely to be present in patients after the administration of a pharmaceutical or diagnostic agent derived from an animal source. For example, administration of rabbit antithymocyte globulin (RATG) as an immunosuppressant can induce production of Human Anti-Rabbit Antibody (HARA) in patients for up to a year. HARA can result in false-positive results in Histoplasma sandwich ELISA tests by reacting with rabbit IgG used as a capture antibody and detector antibody in sandwich immunoassays. For example, Wheat et al. identified false-positive test results in individuals without histoplasmosis in 2003 (as described in Wheat L J, Garringer T, Brizendine E, and Connolly P., “Diagnosis of histoplasmosis by antigen detection based upon experience at the histoplasmosis reference laboratory,” Diagn Microbiol Infect Dis 2002; 43:29-37; Wheat L J. Current diagnosis of histoplasmosis. Trends Microbiol 2003; 11:488-94), incorporated herein by reference in its entirety. One cause for false-positive results was identified in organ allograft recipients who received Thymoglobulin®, as described by Wheat L J, Connolly P, Durkin M et al. False-positive Histoplasma antigenemia caused by antithymocyte globulin antibodies. Transpl Infect Dis 2004; 6:23-7. False-positive Histoplasma antigenemia correlated highly with the presence of Human Anti-Rabbit Antibodies, so called (HARA). This type of interference activity has also been recognized in assays using murine antibodies (HAMA), for example as described in Kricka L J., “Human anti-animal antibody interferences in immunological assays,” Clin Chem 45:942-56 (1999).

Accordingly, there is a need for improved immunoassay tests to identify pathogens, such as Histoplasma fungi, using animal-derived capture and/or detection antibodies in the presence of a human anti-animal antibody. Diagnostic tests for fungal antigens generally, and for Histoplasma antigen in particular, are needed that have a reduced or low level of false positives or false negatives. In particular, improved immunoassay tests for detection of a Histoplasma antigen in the presence of Human Anti-Rabbit Antibody are needed.

### SUMMARY

The present disclosure relates to improved enzyme-linked immunoassay (“ELISA”) kits, procedures and diagnostic methods for identifying one or more fungal antigens, including a Histoplasma capsulatum antigen. Preferred ELISA kits, procedures and methods provide a desirably reduced incidence of false positives and/or false negatives when detecting the antigen. In particular, preferred ELISA assays may have reduced the incidence false positives or negatives caused by the human anti-animal antibodies, including anti-rabbit antibody (HARA). The preferred immunoassays are preferably configured as sandwich (two-site) ELISA immunoassays performed by contacting a sample with a capture antibody bound to form an antigen binding surface on the well of a microtiter plate and contacting the bound antigen to a suitable detector antibody.

The capture antibody preferably comprises an unmodified polyclonal rabbit anti-Histoplasma capsulatum IgG capture antibody, while the detector antibody is preferably a modified polyclonal rabbit anti-Histoplasma capsulatum IgG that does not comprise an F_{c} antibody portion. Most preferably, the polyclonal rabbit anti-Histoplasma capsulatum IgG is the F(ab)′₂ fragment isolated after enzymatic modification (e.g., pepsin) of the IgG antibody to remove the F_{c} portion. The detector antibody is adapted for detection by a suitable method, such as radiologic or optical detection. The detector antibody may be adapted to bind to a reporting molecule to detect the presence of the detector antibody attached to a surface-bound antigen. Antigen-binding results are preferably classified as positive or negative by comparison with a suitable negative control specimen, with readings greater than twice the optical density of the negative control being positive. The optical density of the analyte patient specimen can be divided by the cutoff optical density to obtain results reported in assay units (above 1U being positive for the presence of a particular antigen). Preferably, the results are reported quantitatively in units of mass of antigen per unit volume of analyte. Quantitive reporting of antigen levels can be obtained by comparison of assay results with a calibration curve measured by using control samples of known antigen concentration.

The improved immunoassays preferably include one or more improvements as disclosed herein, relating to improved uniformity in blocking agents, improved detector antibody configurations, improved compositions comprising the detector antibody, and reporting of antigen binding data in a quantitative format. While the embodiments are typically discussed with respect to the Histoplasma antigen, the immunoassay embodiments described herein are applicable to other antigens, including various endemic mycoses fungal antigens.

Preferably, the capture and detection antibodies for ELISA tests are obtained by immunizing a suitable host animal, such as a rabbit, with a mixed vaccine comprising antigens obtained from multiple recent patient isolates. Preferably, capture and detection antibodies are obtained from a rabbit host animal after injection with a vaccine comprising two or more strains of Histplasma antigens, more preferably 2, 3, 4, or 5 strains of Histoplasma antigens. In a second preferred embodiment, non-specific binding to detector plates is blocked by contacting the detector plate with a blocking agent characterized by a reduced incidence of variation in the inhibition of non-specific blocking. In one aspect, the blocking composition is free of bovine serum albumin (BSA). More preferably, the blocking composition is a solution comprising plant-derived proteins. In another embodiment, detection antibodies are preferably combined with an excess of Normal Rabbit Serum (NRS) prior to contact with a capture antibody on an antigen binding surface. The NRS is preferably obtained from a rabbit serum sample selected by a screening method based on the detection of bound detector antibody in the presence of goat anti-rabbit antibody (GARA) with a positive control. Accordingly, an immunoassay preferably comprises the step of preparing a detector antibody composition comprising an animal serum screened for ability to reduce interference with detector antibody binding from a GARA control. In particular, the detector antibody is preferably combined with a serum that reduces the binding of GARA to a capture antibody. Methods of detecting multiple antigens in a single immunoassay are also provided. Notably, the improved immunoassay tests provided herein recognize and detect a cross-reactive galactomannan antigen common to different endemic mycoses. Accordingly, the improved immunoassay may be used to diagnose infections caused all of the endemic mycoses (Histoplasma, Blastomyces, Coccidioides, Paracoccidioides, Penicillium marneffei).

Immunoassay preferably comprises the step of reporting antigen concentration quantitatively in units of concentration (e.g., ng/mL) rather than relative antigen units by comparison of analyte data to a calibration curve. Purified Histoplasma yeast galactomannan is preferably selected as a calibration standard for antigen quantitative reporting of antigen detection.

### BRIEF DESCRIPTION OF THE DRAWINGS

FIG. 1 is.a schematic of a sandwich (two site) immunoassay.

FIG. 2A is a schematic of a false positive result due to binding of an anti-animal antibody in a sandwich immunoassay.

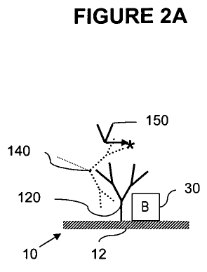

FIG. 2B is a schematic of a false negative result due to binding of an anti-animal antibody in a sandwich immunoassay.

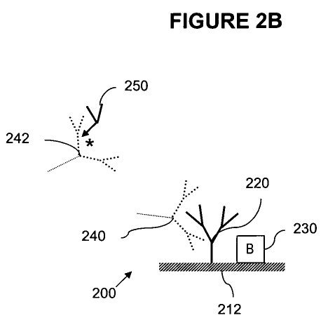

FIG. 3 is a graph comparing the incidence of positive, false positive and false negative cases from an improved sandwich immunoassay with a comparative sandwich immunoassay.

FIG. 4 is a portion of the structure of purified Histoplasma galactomannan antigen.

FIG. 5 is a graph showing the results of cross-reactive detection of Blastomycosis and Coccidioidomycosis antigens in a Histoplasma antigen immunoassay.

FIG. 6A is a calibration curve based on dilutions of a calibration standard comprising known amounts of a galactomannan antigen.

FIG. 6B is a second calibration curve of Example 8.

FIG. 7A is a graph of the readings from an immunoassay conducted on five samples on different days or the same day, expressed in antigen (EIA) units.

FIG. 7B is a graph of the readings from an immunoassay conducted on the same five samples shown in FIG. 7A on different days or the same day, expressed in quantitative units of ng/mL, obtained by use of a calibration curve.

### DETAILED DESCRIPTION

The following definitions are offered to lend clarity to this writing, wherein to the extent that terms presented in this section are defined differently by a dictionary or other sections hereof, then the definition presented in this section shall govern in interpreting this specification and the accompanying claims.

“Ab” is an abbreviation for antibody.

“Ag” is an abbreviation for antigen.

“BALF” is an acronym that stands for bronchoalveolar lavage fluid, which is a physiological specimen that can be tested for presence of Histoplasma antigen using the present invention.

“BSA” is an acronym that stands for bovine serum albumin, which is commonly used as a blocker reagent in ELISAs.

“Coefficient of variation” is an attribute of a distribution, i.e, the standard deviation of the distribution divided by its mean, and is typically expressed as a percentage.

“CSF” is an acronym that stands for cerebrospinal fluid, which is a physiological specimen that can be tested for presence of Histoplasma antigen using the present invention.

“ELISA” is an acronym that stands for enzyme-linked immunoassay; also abbreviated as “EIA,” which is used interchangeably with ELISA.

“F_{c}” means a highly conserved, non-antigen-binding fragment of an immunoglobulin obtained following papain digestion of an immunoglobulin.

“F(ab)₂” means a bivalent antigen-binding fragment obtained following pepsin digestion of an immunoglobulin.

“Fab” means a monovalent antigen-binding fragment obtained following subjecting a F(ab)₂ to a reducing agent.

“HARA” is an acronym that stands for human anti-rabbit antibodies.

“HRP” is an acronym that stands for horseradish peroxidase.

“IgG” is an acronym that stands for immunoglobulin G, which is a class of antibodies found in serum.

“NRS” is an acronym that stands for normal rabbit serum, meaning serum from a non-immunized rabbit, particularly with reference to an antigen derived from H. capsulatum.

“OD” is an acronym that stands for optical density, the subscript of which indicates a wavelength or wavelengths that are used to determine degree of color change, for example, caused by a reaction.

“RIA” is an abbreviation for radioimmunoassay.

“SD” is an acronym that stands for standard deviation, which is a measure of the variability of the distribution of data around the mean.

“UF” is an abbreviation for ultrafiltration, which filtering process of water, usually, generally separates particles sized between 0.1 to 0.005 microns.

Improved Enzyme Radioimmunoenzyme Assays

The present disclosure relates to improved enzyme-linked immunoassay (“ELISA”) kits, procedures and diagnostic methods for identifying a fungal antigen. Preferably, the kits, procedures and methods provide a desirably reduced incidence of false positive and/or false negative indications for detecting one or more enemic mycoses antigen. In particular, preferred ELISA kits and test methods presently disclosed provide a reduction in the incidence false positive or negative indications caused by the human anti-animal antibodies, including human anti-rabbit antibody (HARA). In sandwich (two-site) ELISA immunoassays, human anti-animal antibodies can interfere with antigen detection by binding to capture antibodies and/or detector antibodies. This is particularly problematic in cases where the capture and/or detection antibodies are derived from the same animal as the human anti-animal antibody. For example, patients receiving rabbit anti-thymocyte globulin as an immunosuppressant following organ allograft, for example as a product under the tradename Thymoglobulin®, often produce HARA, which can persist for up to about a year or longer. The undetected presence of HARA in patients can subsequently interfere with ELISA tests using rabbit-derived antibodies, for example by causing false positive or false negative readings. In addition, patients keeping rabbits as pets have been found to produce HARA as well.

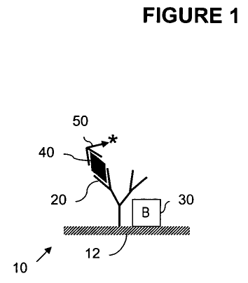

Preferred kits, procedures and methods are adapted to detect one or more antigen indicative of a histoplasmosis in humans, such as the Histoplasma antigen. The immunoassays are preferably configured as a sandwich enzyme immunoassay, as shown in FIG. 1. Preferably, the capture antibody 20 preferably comprises an unmodified polyclonal rabbit anti-Histoplasma capsulatum IgG capture antibody, while the detector antibody is preferably a modified polyclonal rabbit anti-Histoplasma capsulatum IgG that does not comprise the Fc antibody portion. Most preferably, the polyclonal rabbit anti-Histoplasma capsulatum IgG is the F(ab)₂ fragment isolated after enzymatic modification (e.g., pepsin) of the IgG antibody to remove the F_{c} portion. Antigen-binding results are preferably classified as positive or negative by comparison with a suitable negative control specimen, with readings greater than a suitable cutoff, typically about 1.5-3.0 times the optical density of the negative control being positive. The optical density of the analyte patient specimen can be divided by the cutoff optical density to obtain results reported in assay units (above 1U being positive for the presence of a particular antigen).

Improved immunoassays preferably include one or more improvements as disclosed herein, relating to the selection or vaccination of a suitable host animal (such as a rabbit) to obtain capture and detector antibodies, improved uniformity in blocking non-specific binding, broadening the suitable sources of analyte samples, improved detection antibody configurations, collection of antigen binding data in a quantitative format, and a method for identification of false positive results. While the embodiments are typically discussed with respect to the Histoplasma antigen, the immunoassay embodiments described herein are applicable to any suitable antigen, for example by selecting appropriate detector and capture antibodies.

Preparation of Test Plates

In a first embodiment, improvements in immunoassays pertaining to the preparation of test plates for performing ELISA are provided. In particular, improvements in the uniformity of blocking non-specific binding are provided.

According to one aspect of the first embodiment, the capture and detection antibodies for ELISA assays are obtained by immunizing a suitable host animal, such as a rabbit, with a mixed vaccine comprising antigens obtained from multiple recent patient isolates. Preferably, capture and detection antibodies are obtained from a rabbit host animal after injection with a vaccine comprising two or more strains of Histoplasma antigens, more preferably 2, 3, 4, or 5 Histoplasma antigen samples. For example, antibodies obtained from a vaccine obtained using five Histoplasma capsulatum mould isolates obtained from different patients over a 6-7 month period produced an improved antibody, as detailed in Example 9 below. Preferably, the capture and/or detector antibodies are obtained from animals demonstrating about a 40%-50% or greater inhibition of the binding of a F(ab)′₂ detector antibody detected in a test binding assay compared to the binding of the detector antibody in a control binding assay. The test binding assay comprises following steps: providing an antigen binding surface comprising anti-Histoplasma rabbit IgG capture antibody, contacting the antigen binding surface with serum obtained from a vaccinated animal (preferably, a rabbit) and a positive control comprising the Histoplasma antigen in a manner effective to bind the Histoplasma antigen to the capture antibody, contacting the bound Histoplasma antigen with a detector antibody comprising a biotin moiety and the F(ab)′₂ portion of the anti-Histoplasma rabbit IgG antibody separated from (or without) the F_{c} portion, and detecting the bound detector antibody. The test binding assay is the same as the test binding assay, except that the serum is obtained from a non-vaccinated rabbit. The percent-inhibition is defined as the amount of bound detector antibody detected in the test binding assay divided by the amount of bound detector antibody detected in the control binding assay (e.g., ODₜₑₛₜ/OD_{control}). In the results obtained in Example 9, one of five rabbits vaccinated with a single antigen sample showed a percent inhibition of 40% or greater. In contrast, nine out of ten rabbits vaccinated with the five antigen samples showed a percentage inhibition of 40% or greater. Accordingly, detector and captive antibodies are preferably obtained from an animal vaccinated with two or more antigen samples, preferably from two or more Histoplasma antigens obtained from patient samples that are less than about 2 years old. Preferably, anti-Histoplasma IgG detector antibodies may be contacted with an enzyme to remove the F_{c} portion

Once isolated from the animal, the capture antibodies are attached to an immunoassay detection plate by any suitable method. Such attachment can be accomplished by the physical adsorption of the capture antibody to the surface, by action of van der Waals forces or hydrophobicity or the like. It is generally known among those skilled in the art that proteins generally, and certainly antibodies, have an affinity for plastic or glass surfaces, which are preferred surfaces used in the context of the present invention. Other preferred surfaces include polymers, both natural, such as cellulose or chitin and the like, and synthetic, such as nylon and the like. Most preferred surface used in the context of the present invention is a plastic surface. One could also attach the capture antibody to the surface by use of reactive groups that are themselves attached to the surface and that react covalently to the capture antibody.

The capture antibody is preferably an antibody derived from a first animal, such as a rabbit, wherein the antibody binds to at least one Histoplasma antigen. A particularly preferred capture antibody is a rabbit-derived IgG antibody for H. capsulatum. Optionally, the capture antibody can be modified by removing the F_{c} or F(ab)′₂ portion, as described in detail with respect to the detector antibody below. The antigen binding surface can have any suitable concentration of the capture antibody.

In a second aspect of the first embodiment, non-specific binding to an antigen binding surface is blocked by contacting the detector plate with a blocking composition. Preferably, the blocking composition is substantially free of bovine serum albumin (BSA). The antigen binding surface comprising a capture antibody is typically contacted with a blocking agent prior to contact with an analyte sample for antigen-binding analysis. The blocking agent is desirably provided as an excess of a suitable compound that will attach to the antigen binding surface in a manner that substantially reduces or prevents non-specific antigen binding (i.e., antigen binding to the surface other than to the bound capture antibody). Preferably, the blocking agent does not itself attract specific or nonspecific attraction of the antigen of interest or antibody directed thereto. As described in detail in Example 2, certain low and high positive controls were not detected in immunoassays in the presence of certain BSA plate blocking compositions. Comparable immunoassay data provided in Tables 1-4 indicated variability in immunoassay performance when using different BSA plate blocking compositions. A coefficient of variation of about 0.23-0.24% was observed for the high positive, low positive and negative control samples using BSA plate blocking compositions tested in Example 2. The coefficient of variation is obtained by dividing the standard deviation by the mean of a series of assay measurements. Lower variability was observed when performing comparable immunoassays with preferred plate blocking compositions in Example 3, as indicated by the data of Table 5. As described in Examples 2 and 3, particularly preferred blocking compositions have a coefficient of variation of less than 0.20% for a sample size of 10 or more, and preferably less than 0.15, 0.10 or 0.05. Preferably, the blocking composition is a solution comprising plant-derived proteins, such as Starting Block™ (Pierce Biotechnology, Inc.).

After contacting the antigen binding surface with the blocking composition, an analyte is placed in contact with the antigen binding surface. The analyte is typically a physiological specimen containing an unknown amount of an antigen, a positive control known to contain the antigen, or a negative control known to not contain the antigen. The physiological specimen used in the context of the present invention is any specimen that may be collected from a patient. Preferably, the analyte is either a fluid when removed from the patient or macerated or soaked in a physiological saline buffer. Preferably, the physiological specimen is selected from the group consisting of serum, urine, cerebrospinal fluid, bronchoalveolar lavage fluid, pleural fluid, pericardial fluid, peritoneal fluid, synovial fluid, ocular fluid, and abscess contents.

Improved Antibody Compositions

In a second embodiment, improvements in immunoassays pertain to improved detection antibody configurations permitting a reduction in false positive readings and/or an increase in the number of suitable sources of analyte samples. In particular, detection antibodies are preferably combined with an animal serum that is selected based on a serum screening process to identify serum samples characterized by the ability to block or reduce interference by agents that are capable of causing false positives. For example, rabbit serum can be screened to identify serum from rabbits that reduces interference by Goat Anti-Rabbit Antibody (GARA) (e.g., during serum screening described below) and HARA (e.g., during clinical testing) in ELISA detection.

In a first aspect of the second embodiment, detection antibodies are preferably combined with an excess of animal serum, such as Normal Rabbit Serum (NRS), prior to contact with a capture antibody on an antigen binding surface. The animal serum is preferably selected by a screening method based on the use of an anti-animal antibody as a positive control. The animal serum is preferably obtained from a different animal of the same species as the source of the detector and/or capture antibodies. Most preferably, the serum is derived from a first rabbit, screened by use of a goat anti-rabbit antibody. Surprisingly, significant variation was observed in the ability of NRS obtained from different rabbits to reduce false positive indications from the presence of goat anti-rabbit antibody (GARA) in sandwich immunoassays using the polyclonal anti-Histoplasmosa rabbit IgG capture antibodies and the corresponding biotinylated F(ab)′₂ fragment. Variation in the ability of NRS obtained from different rabbits to block false positive indications caused by GARA was evaluated in a series of immunoassays performed on a high and low positive Histoplasma antigen control and a goat anti-rabbit antibody control, as described in Example 4. Table 9 of Example 4 provides results from these immunoassays performed using the F(ab)′₂ fragment of the anti-Histoplasmosa rabbit IgG as a detector antibody in an NRS diluent (ca. 1 ppm detector antibody) obtained from 26 different rabbits. Significant variation in the ability of NRS samples to block or reduce GARA interference, with 6 of the 26 rabbit NRS samples failing to reduce interference from GARA activity, and three samples reducing detection of the high positive control. Therefore, selecting NRS samples that exhibit a high optical density for the High Positive control, or more preferably a minimal optical density for GARA activity in Table 9 are particularly desirable for combination with a detection antibody.

Accordingly, an immunoassay preferably comprises the step of preparing a detector antibody composition comprising an animal serum screened for ability to reduce interference with detector antibody binding from a GARA control. In particular, the detector antibody is preferably combined with a serum that reduces the binding of GARA to a capture antibody. Preferably, the binding of the detector antibody to the bound antigen is not reduced by the presence of the serum. More preferably, the serum reduces the binding level of GARA to less than 3.0-times, more preferably less than 1.5-times, the detected level of detector antibody binding to a capture antibody in the presence of a negative control. The serum is preferably derived from a different animal of the same animal species as the source of the detector antibody and/or the capture antibody. Normal Rabbit Serum is one particularly preferred serum. The immunoassay can therefore comprise the step of performing a serum screening assay to identify serum samples that desirably reduce false positives, as indicated by the ability of the serum to increase detector antigen binding to a capture antibody in the presence of GARA. A serum screening assay preferably comprises one or more of the following steps: (a) providing a serum sample, (b) combining the serum with a detector antibody to form a detector antibody solution, (c) providing immunoassay test plates having a capture antibody attached thereto, (d) contacting the detector antibody solution with separate immunoassay test plates in the presence of a negative control, a positive control or a control comprising GARA (“GARA control”), (e) separately detecting the binding of the detector antibody to the immunoassay test plate in the negative control, the positive control and the GARA control, and (f) selecting the serum for inclusion in a detector antibody composition if binding of the detector antibody to the GARA control was reduced in the presence of the serum. Preferably, the serum included with the detector antibody also permits binding of the detector antibody to a bound antigen (true positive) at more than 50% greater level than in the negative control. Step (d) can be modified by replacing the positive control with both a high positive control (such as a 1:10 dilution of a control positive sample (e.g., urine)) and a low positive control (such as a 1:2,000 dilution of the high positive control sample).

In a second aspect of the second embodiment, the detection antibody is a modified IgG antibody that does not comprise the crystalline F_{c} domain. Immunoglobulin structure consists of an antigen-binding domain (“F(ab)′₂”) and a highly conserved crystalline domain (“F_{c}”), which can be separated by proteolytic digestion with papain to obtain the F_{c} fragment or pepsin to obtain the F(ab)′₂ fragment. The F_{c} fragment has a very similar amino acid sequence among all immunoglobulin G (“IgG”) molecules of at least the same species; in contrast, the F(ab)′₂ portion has both hypervariable as well as highly conserved regions when compared from antibody to antibody. The F(ab)′₂ portion comprises two F(ab) fragments paired due to certain disulfide bonds that serve to form the F(ab)′₂ structure. Accordingly, a preferred detector antibody used in the context of the present invention is a F(ab) or F(ab)′₂ fragment. Another preferred antibody derivative would retain the hypervariable regions found on the F(ab) structure but would have removed therefrom, or have masked, the constant regions found thereon. The capture or detector antibody can be a monoclonal, or a polyclonal, or a cloned nucleic acid that encodes the recognition site of the antibody of interest, from the same or a different species of animal than the capture antibody. Preferably, the antibodies used are monoclonal or a polyclonal antibodies; more preferably, the antibodies are polyclonal antibodies; most preferably, the antibodies are polyclonal preparation of an immunoglobulin G antibody.

In general, the capture antibody, the detector antibody, and the animal serum can be derived from the same or different animals that have an immune system, which animals are individually of the same or different species. In preferred embodiments, the species of the animal in which the capture antibody is raised is preferably derived from a polyclonal preparation from rabbit origin. The detector antibody is preferably of rabbit origin as well. In embodiments where the detector antibody is administered in combination with a screened animal serum, the serum is typically derived from a different animal of the same species, preferably a rabbit. An alternative antibody source is of mouse origin, such as a monoclonal detector antibody directed at an epitope of sufficient affinity for the monoclonal antibody that the detector antibody binds to captured Histoplasma antigen on the surface.

The detector antibody preferably recognizes the antigen of interest and is adapted to bind to a reporter element. Optionally, a portion of the detector antibody itself can be detected. Typically however, a portion of the detector molecule is capable of high-affinity binding to a reporter molecule that can be readily detected. For example, the detector antibody can be adapted to bind to a reporter molecule by combining the detector antibody with a biotin moiety to form a high affinity link with a reporter element comprising a streptavidin moiety. Other linking means for joining a reporter element to the detector antibody include, without limitation, sulfosuccinimidyl-4-N-maleimidomethyl-cyclohexane-1-carboxylate (Sulfo-SMCC), sulfosuccinimidyl-6-3′-2-pyridyldithio-propionamido-hexanoate (Sulfo-LC-SPDP), N-maleimidobutyrloxy-sulfo-succinimide ester (Sulfo-GMBS), and the like; two complementary segments of DNA; and a lectin and an appropriate sugar. Unbound detection antibodies can be removed by washing the surface with a wash solution, commonly a neutral saline solution. The wash step at this point in the ELISA protocol is optional depending on whether the protocol is a heterogeneous or homogeneous format.

After contacting a suitable detection enzyme composition with an antigen binding surface under conditions permitting the detection antibody to bind to the surface bound antigen, a composition comprising a reporter element molecule can be contacted with the bound detection antibody. The reporter element molecule is preferably adapted to bind to the detector antibody with a high affinity. For example, when a biotinylated detector antibody is used, a streptavidin-bound reporter element molecule such as horseradish peroxidase can be used. The reporter element composition is added under conditions to permit, or preferably to promote, binding of the detector antibody to the reporter element molecule to form a reporter-conjugated matched pair molecule. Subsequently, unbound reporter-conjugated matched pair molecule can be removed by washing the surface with the same or similar wash solution. This wash step is particularly preferred for either hetero- or homogeneous ELISA formats, as the enzyme conjugated to the matched pair (e.g., the biotin-streptavidin combination) is the signal generator by which the ELISA test is assessed, as further described below.

Preferably, the reporter element-conjugated matched pair component includes an enzyme or a tag that generates a signal by itself (in the case of a fluorescent or radioactive tag) or in the presence of a substrate (in the case of certain enzymes), which signal is commonly a pigment, or visible light, or fluorescence, or radioactivity. Preferred enzymes used in the context of the present invention include, without limitation, a peroxidase, alkaline phosphatase, beta-galactosidase, chloramphenicol acetyl transferase, and/or a luciferase (e.g., that of renilla or a firefly). Preferred substrates for such enzymes include, without limitation, luciferin, tetramethylbenzidine, diethanolamine, p-nitrophenol phosphate (PNPP), 2,2′-azino-bis [ethylbenzthiazoline-6-sulfonic acid] (ABTS), o-phenylenediamine dihydrochloride (OPD), 2-Nitrophenyl-b-D-galactopyranoside (ONPG), 4-Nitrophenyl-b-D-glucuronide (NPG). Preferred dyes, fluorescent tags, metal tags, radioactive tags, and the like, include: fluoroscein, rhodamine, Texas Red, Cy dyes, R-phycoerythrin, gold, PBXL, magnetic microparticle, and latex microparticle, each of which can be covalently linked to a component of either component of the matched pairs. The method preferably further includes detecting a signal, which includes any or all of detecting or measuring light or radioactive emission, dye generation, color change, magnetic or metallic bound components, light scattering, and the like.

Improved Method for Detecting Histoplasmosis Antigen

In a third embodiment, a particularly preferred immunoassay method and kit for detecting a Histoplasma antigen are provided. The method permits detection H. capsulatum in the presence of HARA with desirably low incidence of false negative readings. The kit comprises an ELISA microtiter plate comprising an antigen binding surface, and a detection antibody composition. Most preferably, a kit comprises a screened animal serum and a detector antibody comprising a modified IgG antibody. The modified IgG detector antibody preferably does not comprise a F_{c} fragment, and can be a F(ab)′₂ fragment. The detector antibody is preferably adapted to couple with a reporting element, for example by a biotin-streptavidin linkage. The kit may further comprise control samples for high and low positive readings, as well as a negative sample. Control samples may be obtained from clinical isolates or other sources. A blocking reagent having a desirably low coefficient of variation can also be included, such as a blocking agent substantially free of BSA. A set of suitable reporting reagents, such as HRP, TMB and H₂SO₄, may also be included to provide a means for detecting bound detector antibody.

To form the microtiter plate, a capture antibody is obtained from a suitable animal, such as a rabbit, obtained after vaccinating the animal with two or more, preferably 2-5, different strains of Histoplasmosis antigen in any suitable manner (see, e.g., Example 9). The capture antibody is preferably an unmodified anti-Histoplasmosis IgG rabbit antibody, but may also be a modified IgG antibody having the F_{c} or F(ab)′₂ portion truncated or removed. The capture antibody can be immobilized on an ELISA microtiter plate by any suitable method to form a detection surface. Next, the detection surface is contacted with a suitable blocking medium. Preferably, a blocking agent is selected having a coefficient of variability of less than 0.2% in a manner permitting binding of the blocking medium to the detection surface to form an antigen binding surface in the presence of an anti-animal antibody such as GARA or HARA. The blocking medium is preferably substantially free of BSA, and may contain one or more plant-derived protein.

A detection antibody composition preferably comprises a screened animal serum and a modified anti-Histoplasmosis IgG rabbit antibody. The screened animal serum is selected to provide a reduction in Goat Anti-Rabbit Antibody (GARA) interference with the binding of the detection antibody to the capture antibody. Preferably, the screened animal serum is Normal Rabbit Serum that reduces the binding of the detector antibody to the GARA. The screened animal serum and the detection antibody are typically, but not necessarily, obtained from different animals of the same species, preferably rabbits. The detection antibody is preferably modified by removing or separating the F_{c} portion from the F(ab)′₂ portion to reduce the incidence of false positive readings. Preferably, the detection antibody is the F(ab)′₂ fragment of the anti-Histoplasmosis IgG rabbit antibody used as the capture antibody. The detection antibody is preferably adapted to bind to a reporter element, such as horseradish peroxidase, for example by attaching a biotin moiety to the detection antibody and a streptavidin moiety to the reporter element molecule. Finally, the detection antibody can be combined with the screened animal serum in any suitable manner to provide a detection antibody composition adapted for contacting an antigen binding surface.

Samples containing positive controls, negative controls or samples for analysis can be contacted with the antigen binding surface in the microtiter ELISA plate in any suitable manner permitting antigen binding to the capture antibodies. Optionally, unbound antigen can be rinsed from the antigen binding surface. Subsequently, the detector antibody composition can be added to the microtiter plate in a manner permitting the detector antibody to bind to surface bound antigen. Unbound detector antibody can be removed from antigen binding surface and a reporter element can be added to the microtiter plate to attach the reporter element to detector antibodies attached to bound antigen. The reporter element can be stabilized in the blocking composition, preferably comprising plant-derived proteins. Finally, the reporter element can be detected using any suitable method, including radiodetection or optical density detection.

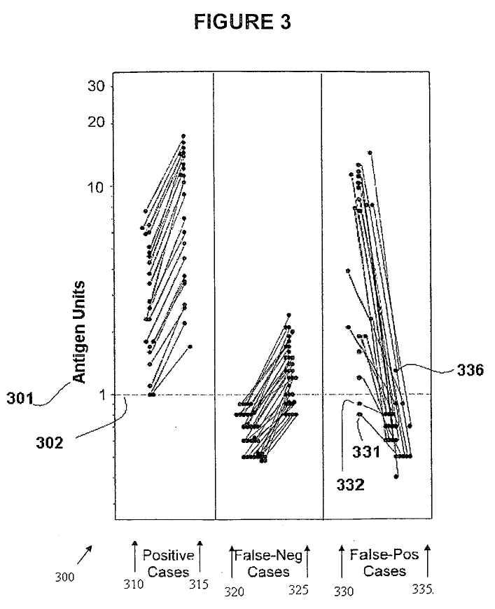

Referring to FIG. 3, results of this improved method are compared to methods of Example 1. The graphs 300 show a comparison of the sensitivity in disseminated histoplasmosis cases in patients with acquired immunodeficiency syndrome who had been treated with fluconazole. Data is reported in antigen units 301, with data points above the line 302 corresponding to 1 antigen unit (optical density divided by the cutoff reading) are positive. Data plotted as “Positive Cases” compares antigen levels in the urine of patients with positive results in the assay of Example 1 (data in column 310) with results obtained using the immunoassay of the third embodiment described above (data in column 315). Data plotted as “False-Negative Cases” compares antigen levels in the urine of patients with disseminated histoplasmosis that had become negative following fluconazole treatment in the same clinical trial using the assay of Example 1 (data in column 320) with results obtained using the third embodiment immunoassay described above (data in column 325). Data plotted as “False-Positive Cases” compares antigen levels in the serum of organ transplant patients with false-positive cases caused by HARA using the assay of Example 1 (data in column 330) with results obtained using the immunoassay described in the third embodiment above (data in column 335). All but two data points (data points 331, 332) were false using the assay of Example 1 (data in column 330), while only 1 data point (336) remains a false positive using the immunoassay test of the third embodiment above (data in column 335).

Immunoassay Cross-Reactivity

In a fourth embodiment, methods of detecting multiple antigens in a single immunoassay are provided. Notably, the improved immunoassay tests provided herein recognize and detect a cross-reactive galactomannan antigen common to different endemic mycoses. Accordingly, the improved immunoassay, such as an immunoassay according to the third embodiment, may be used to diagnose infections caused by any of the endemic mycoses (Histoplasma, Blastomyces, Coccidioides, Paracoccidioides, Penicillium mameffei).

In particular, methods of detecting antigens that share at least one antigen structure with H. capsulatum are provided. Preferably, immunoassays for detecting antigens comprising galactomannan are provided that comprise the steps described with respect to assay of the third embodiment. Yeast cell wall galactomannan is believed to be the antigen detected in the Histoplasma antigen assay. Evidence for this includes stability at 100° C., resistance to proteases, susceptibility to glycosidases and sodium periodate (See, Wheat, L. J., R. B. Kohler, and R. P. Tewari, “Diagnosis of disseminated histoplasmosis by detection of Histoplasma capsulatum antigen in serum and urine specimens,” N. Engl. J. Med. 314:83-88 (1986), incorporated herein by reference in its entirety), and affinity to concanavalin A.

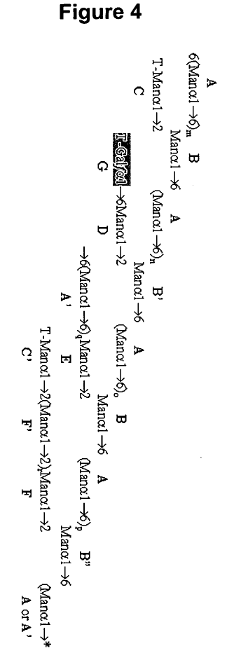

FIG. 4 describes the structure of the major portion of the Histoplasma yeast galactomannan. The structural features represented by residues B, B′, C, G, and D are shared in common with galactomannans from Paracoccidioides brasiliensis (as described in Azuma I, Kanetsuna F, Tanaka Y, Yamamura Y, and Carbonell L M., “Chemical and immunological properties of galactomannans obtained from: Histoplasma duboisii, Histoplasma capsulatum, Paracoccidioides brasiliensis and Blasomyces dermatitidis,” Mycopatholog Mycolog Appl, 54:111-25 (1974)); this is believed to include indications of short stretches of→2Manα1→2 residues. However, the structures differ in several respects. Neither the yeast nor mycelium form P. brasiliensis polysaccharides features unbranched backbone→6Manα1→6 residues; most importantly, the H. capsulatum yeast form galactomannan does not have any detectable β-Galf1→residues, which were found in the corresponding yeast form P. brasiliensis polysaccharide (although not in the mycelium form of that fungus). Terminal (1→5)-α-D-galactofuranose was identified in the major portion of the Histoplasma galactomannan (see G in the figure above), and is likely the epitope detected in the antigen assay. Thus, one particularly preferred method provides immunoassays for detecting antigens comprising the T-Galfα1 (G) antigen in FIG. 4.

Purified galactomannan demonstrated reactivity by immunoblot with rabbit antibodies to H. capsulatum used for antigen detection, producing a high molecular weight diffuse smudge (41-110 Kda), similar to that seen with the antigen detected in the urine of patients with histoplasmosis. NMR, monosaccharide analysis, and methylation linkage data suggest that a major portion of the H. capsulatum galactomannan can be described by the general structure below (no particular order of side-chain substitutions C-F is implied).

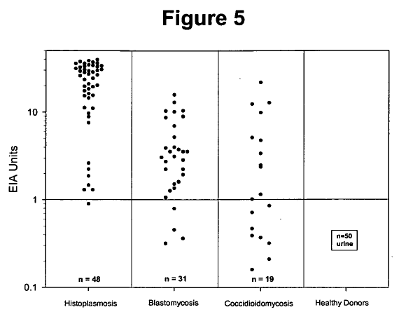

Surprisingly, specimens from patients with histoplasmosis may cross-react in an Aspergillus galactomannan Elisa assay. Antibodies to Aspergillus galactomannan are not believed to cross-react with the galactomannan produced by the endemic mycoses. Cross reactions with endemic mycoses have not been reported despite over 10 years of experience with the Aspergillus antigen assay. However, as shown in FIG. 5, cross-reactive antigen detection in the Histoplasmosa antigen assay was identified from patients with blastomycosis and coccidioidomycosis. Each data point represents a single patient, with results expressed in antigen (EIA) units. Results above 1.0 antigen units are positive. Positive results were noted in nine of 20 (45%) serum specimens and six of ten (60%) bronchoalveolar lavage fluid specimens containing elevated levels of Histoplasma antigen. These findings suggest that Histoplasma and Aspergillus galactomannan share some antigenic characteristics, although less than the endemic mycoses. Cross-reactivity between Histoplasma and Aspergillus galactomannan has not been previously observed.

(1→5)-β-D-galactofuranose residues on the side chains of galactomannan (as disclosed, for example, in Bernard M and Latge J P., “Aspergillus fumigatus cell wall: composition and biosynthesis,” Med Mycol., 39 Suppl 1:9-17 (2001)) are believed to be the immunodominant cell wall polysaccharides of Aspergillus, as shown in inhibition studies (as described in Bennett J E, Bhattacharjee A K, and Glaudemans C P., “Galactofuranosyl groups are immunodominant in Aspergillus fumigatus galactomannan,” Mol Immunol 22:2514 (1985); as well as in Notermans S, Veeneman G H, van Zuylen C W, Hoogerhout P, and Van Boom J H, “(1-5)-linked beta-D-galactofuranosides are immunodominant in extracellular polysaccharides of Penicillium and Aspergillus species,” Mol Immunol, 25:975-9 (1988)). Furthermore, acid hydrolysis, which removes the galactofuranose side chains, has been shown to destroy the antigen properties of galactomannan (Reiss E and Lehmann PF. Galactomannan antigenemia in invasive aspergillosis. Infect Immun, 25:357-65 (1979); and Haido R M, Silva M H, Ejzemberg R et al., “Analysis of peptidogalactomannans from the mycelial surface of Aspergillus fumigatus,” Med Mycol, 36:313-21 (1998)). (1→5)-α-D-galactofuranose is believed to be a key epitope for Histoplasma, compared to the (1→5)-β-D-galactofuranose for Aspergillus. Differences in these galactofuranosyl epitopes appear to account for the low-level cross-reactivity observed in histoplasmosis and aspergillosis, intermediate cross-reactivity in coccidioidomycosis, and high-level cross-reactivity in blastomycosis, paracoccidioidomycosis, and penicilliosis mameffei.

Quantitative Immunoassay Reporting

In a fifth embodiment, a method of quantitative reporting of immunoassay results is provided. Typically, the detection of antigen levels in immunoassays are expressed semiquantitatively by comparison of the amount of bound detector antibody in the presence of an analyte antigen with a cutoff derived by multiplication of the negative control by a factor typically ranging from 1.5-3.0. Accordingly, the amount of antigen in a sample is typically reported in antigen units (for example, by dividing the detected antigen signal by the cutoff or the negative control). However, due to day-to-day variability in antigen assay measurements, it is usually necessary to test a prior sample along with the current sample in the same assay to assess the change in antigen during treatment. In contrast, methods for reporting of antigen concentration by comparison to a calibration curve are provided herein, wherein antigen concentration is provided in units of concentration (e.g., ng/mL) rather than antigen units. Dilutions of a urine pool from patients with histoplasmosis, determined to contain known amounts of Histoplasma galactomannan, by comparison to purified Histoplasma yeast galactomannan, is preferably selected as a calibration standard for antigen quantitative reporting of antigen detection.

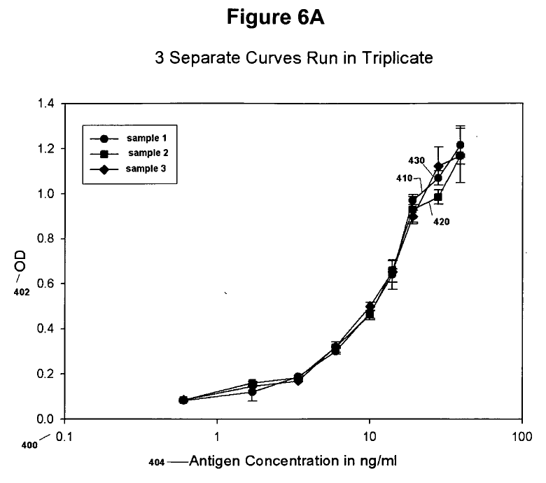

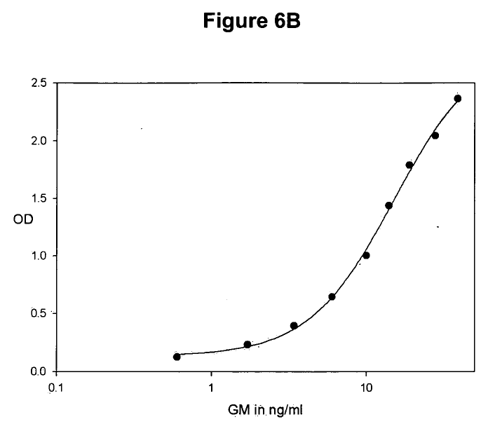

FIG. 6A is a calibration standard curve 400 prepared from three samples of urine from patients with histoplasmosis, showing the optical density 402 as a function of antigen concentration 404 for a first sample 410, a second sample 420 and a third sample 430. Each of the three samples was prepared from a pool of urine specimens containing high levels of Histoplasma antigen. Urine specimens were first screened for cross-reactivity in the Platelia Aspergillus galactomannan antigenemia assay (BioRad), and those that were positive were excluded from the calibrator pool. Multiple dilutions of the calibrator pool were prepared, and the antigen content of each calibrator was determined by comparison to known concentrations of the purified galactomannan, at concentrations of 39, 28, 19, 14, 10, 6, 3.4, 1.7, and 0.6 ng/mL (i.e., from 0.6 ng/ml to 39 ng/ml). Accordingly, urine calibrators were assigned ng/ml concentration values. Error bars on each curve indicate the standard deviation for each of the three samples. The standard curve was highly reproducible when determined on multiple occasions, as evidenced by the closeness of the three curves in FIG. 6A. A second calibration curve in FIG. 6B is discussed in Example 8.

Immunoassay results obtained from the immunoassay of the third embodiment were classified as positive or negative by comparison of the optical density of the test specimen to that of a negative control specimen. Galactomannan antigen concentration of specimens determined to be posited by comparison to the nitrogen control was determined by comparing the optical density of the test specimen to that of the calibration curve standards, and results were expressed ng/ml. Specimens with results exceeding the cutoff for the assay but less than the lowest standard were reported as positive, less than 0.6 ng/ml, and results higher than the 39 ng/ml calibrator as ≧39 ng/ml.

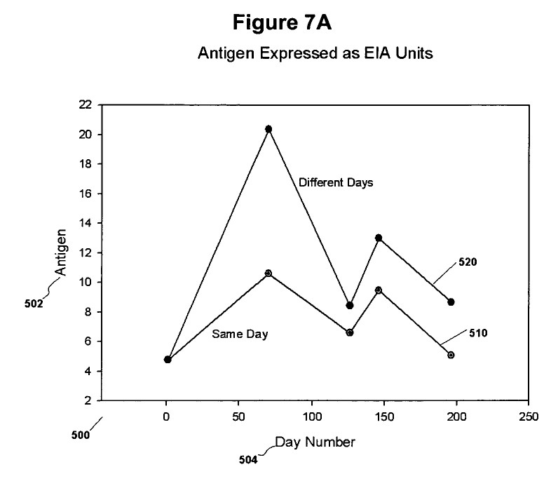

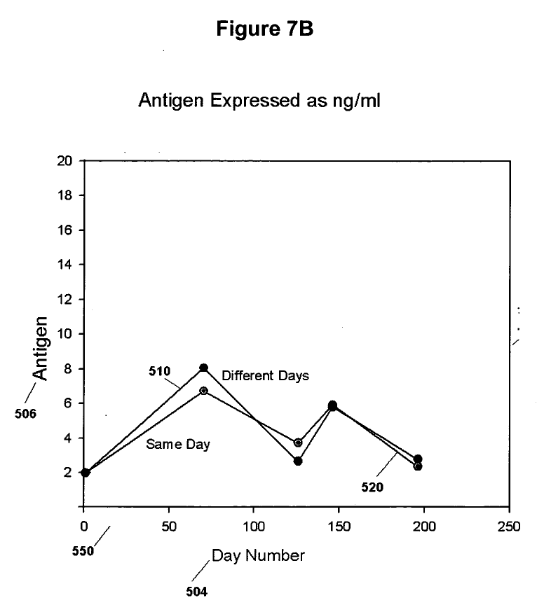

Immunoassay tests comprising the step of quantitatively reporting results using a suitable calibration curve, such as the curve 400 of FIG. 6A, provided reduced variability compared to antigen level reporting by antigen units. FIG. 7A shows the results from immunoassay testing for a Histoplasmosis antigen on five different specimens each obtained from a single patient on five different days over a 200-day period. After collection of the five specimens, the same Histoplasmosis immunoassay was performed on each sample on the same day to generate a first curve 510, and then each sample was again tested on different days with the same assay to generate a second curve 520. FIG. 7A shows the results of these immunoassay tests of the same five patient samples expressed in antigen (EIA) units. FIG. 7B shows the same results reported quantitatively in ng antigen/mL, obtained by comparison to a calibration curve, showing much closer similarity between the first curve 510 (from testing each of the five samples on different days) and the second curve 520 (from testing each of the five samples on the same day). Results from the same versus different days agreed more closely in the quantitative assay, expressed as ng/ml (FIG. 7B), than in the semi quantitative assay, expressed as EIA units (FIG. 7A).

### EXAMPLES

The following examples illustrate the invention, but are not to be taken as limiting the various aspects of the invention so illustrated. Chemicals and reagents not otherwise indicated as to commercial source may be obtained from the Sigma Chemical Co. (St. Louis, Mo.).

### Comparative Example 1

This example illustrates an ELISA diagnostic test directed at Histoplasma antigen, as set forth by Durkin et al., J. Clin. Microbiol. 35(9):2252-55 (1997), whereat the ELISA test was compared to results from the same specimens that had previously been subjected to analysis by a standard radioimmunoassay diagnostic test for the same antigen.

Experimental phvsiological specimens and storage thereof. Urine specimens were collected from histoplasmosis patients from 1988 to 1992, a period of high incidence of this disease in Indianapolis, and were stored at 4° C. Previous RIA results for these patients were reviewed, and specimens were chosen to provide a range of results from negative to high positive. Urine specimens from 45 non-AIDS patients (16 with disseminated histoplasmosis, 22 with pulmonary histoplasmosis, 5 with cavitary histoplasmosis, and 2 with pericarditis) and 41 AIDS patients (40 with disseminated histoplasmosis and 1 with pulmonary histoplasmosis) were used to compare the ELISA method of antigen detection to that of the prior radioimmunoassay.

Control phvsiological specimens for testing the standard ELISA. Control physiological specimens used to test the ELISA were urine specimens from the following individuals:
20 healthy laboratory personnel;
28 patients with fungal infections other than H. capsulatum, specifically
   12 with Candida
   three with Aspergillus
   two with Blastomyces dermatitidis
   one with Paracoccidioides brasiliensis
   one with Coccidioides immitis
   three with Cryptococcus neoformans
   two with Pneumocystis carinii, and
   four with miscellaneous fungal infections;
24 patients with urinary tract infections, specifically
   12 with Escherichia coli
   five with Klebsiella pneumoniae
   one with Proteus mirabilis
   four with Staphylococcus spp.
   one with streptococcus group D, and
   one with Citrobacter freundii; and
24 patients with nonfungal pneumonia, specifically
   20 with Streptococcus pneumoniae
   two with Haemophilus influenzae
   one with Mycobacterium tuberculosis, and
   one with Nocardia sp.

The ELISA method. Unless otherwise indicated, reagents and chemicals for making buffers and solutions of antibodies or enzymes or substrates were purchased from Pierce Biotechnology, Inc., Rockford, Ill. The wells of Immulon-2 microtiter plates (rigid plates. whose wells have flat bottoms; Dynatech Laboratories) were coated with 100 μl of an immunoglobulin G (IgG) fraction of rabbit anti-Histoplasma serum (the capture antibody) in 0.01 M Tris-HCI (pH 7.0) (0.01 M Tris), incubated at 37° C. for 1 hour, and washed with phosphate-buffered saline (pH 7.2) containing 0.05% polyoxyethylene(20)sorbitan monolaurate (sold under the tradename “Tween® 20” by EMD Biosciences Inc., San Diego, Calif.). The wash buffer was made fresh daily. Two hundred microliters of a plate blocking buffer (i.e., 5% bovine serum albumin in 0.01 M Tris) was added to each well, and the plate was incubated and washed as described above.

Next, 100 μl of undiluted urine was added to each well, and the plate was incubated and washed as described above. The wells were incubated with 100 μl of rabbit anti-Histoplasma IgG conjugated to biotin (the detector antibody) in 0.1 M Tris-HCl (pH 8.0) (0.1 M Tris) and washed as described above.

Finally, Histoplasma antigen adhering to the solid-phase antibody was measured by adding 100 μl of streptavidin-horseradish peroxidase in 0.1 M Tris-5% bovine serum albumin to each well. The plate was incubated and washed as described above. Peroxidase substrate (tetramethylbenzidine; Cappel Research Products, Durham, N.C.) was dissolved in a citrate buffer (0.1 M citric acid; pH 4.0), and 100 μl was added to each well. Color development was stopped by the addition of 100 μl of 1.0 M H₂SO₄ to each well, and the optical density of the plate was read on a microplate-reading spectrophotometer (e.g., V-Max® sold by Molecular Devices Corporation, Sunnyvale, Calif.) at a wavelength of 450 nm (“OD₄₅₀”). Results that were 50% higher than the mean value for normal, negative samples were considered positive. All results were divided by 1.5 times the mean value for the normal urine samples and were expressed as RIA units or EIA units, as appropriate.

The RIA method. Alternatively, a detector antibody can include a radioimmunoassay reporter element such as ¹²⁵I to detect the presence of the detector antibody bound to the antigen binding surface. For example, the radioimmunoassay (“RIA”) method described by Wheat et al., New England J. of Medicine 314:83-8 (1986) measures bound antigen with the same rabbit anti-Histoplasma IgG that is used in the ELISA, but it was labeled with ¹²⁵I instead of biotin. The wells were washed between all steps with 0.15% NaCl.

Reproducibility. The reproducibility of the ELISA method was examined by testing specimens from 34 histoplasmosis patients and 10 negative specimens in two consecutive assays within a period of one week. Results from each test date were plotted against each other, and a linear regression line and correlation coefficient were calculated using standard procedures. The correlation coefficient was determined to be 0.995, the P value was<0.0001, the intercept was 0.155, and the slope was 0.990; meaning that there was an excellent correlation demonstrated by linear regression analysis, thus indicating a high degree of reproducibility.

Sensitivitv and specificity. A total of 86 urine specimens from patients with histoplasmosis and 96 control specimens were tested. Histoplasma antigen was detected by both EIA and RIA in 61 of the 86 specimens (71%) from histoplasmosis patients and one of the 96 (1%) control specimens. Both systems detected antigen in a specimen from a control patient with paracoccidioidomycosis, indicating the presence of a cross-reacting antigen. Both the RIA and the EIA detected Histoplasma antigen in 50 of 56 (89%) patients with disseminated histoplasmosis.

Among patients with AIDS and disseminated disease, antigen was detected in 38 of 40 (95%) patients, while among patients with disseminated histoplasmosis without AIDS, antigen was detected in 12 of 16 (75%) patients. Among patients with nondisseminated cases of infection, antigen was detectable in 11 of 30 (37%) patients. Among those patients with nondisseminated histoplasmosis, antigen was detected in 10 of 23 (43%) patients with acute pulmonary disease, one of five (20%) patients with cavitary histoplasmosis, and zero of two patients with pericardial disease.

Correlation of EIA and RIA. The results of the RIA and EIA were compared by linear regression analysis using standard techniques. There was a good correlation between the two methods, with a correlation coefficient of 0.974 and a P value of <0.0001. The slope was 0.915, and the intercept was −0.013. The RIA system had a greater range of results, with the highest result being 27.0 units. In contrast, for the same urine sample the result was 20.1 units by the EIA.

Conclusion. Accordingly, the ELISA with a biotin-conjugated antibody is believed to be as sensitive and specific as the RIA, and thus presented no improvement in false positive and false negative results. Antigen was detected in 89% of patients with disseminated cases of infection and 37% of patients with non-disseminated cases of infection. Antigen was detected in 95% of the patients with disseminated cases of infection and AIDS, whereas antigen was detected in 75% of those with disseminated cases of infection but without AIDS.

The false-negative results were highest for non-disseminated cases of histoplasmosis (about 63% false negative), substantially less for disseminated cases (about 25% false negative), and least for disseminated cases where the patients were also afflicted with AIDS (about 5% false negative).

### Example 2

Variability was observed in the results from a series of Histoplasma ELISA tests conducted in accordance with the procedure set forth in Example 1, using various blocking agents comprising Bovine Serum Albumin (BSA).

Specifically, the Histoplasma antigen assay of Example 1 failed to detect the low positive and high positive controls or patient specimens on three consecutive days. The only reagent that differed from previous and subsequent tests that properly detected high and low control samples was the source of the plate blocking buffer, i.e., the 5% solution of bovine serum albumin (“BSA”) used in the ELISA assay. The data in question is provided in Table 1 below showing 9 assays performed from July 1 (7/01) through September 27 (9/27). Each assay was performed according to the procedure in Example 1, using the lots of Sigma blocking agent indicated in Table 1. Notably, the high positive and low positive controls were not detected in the three assays performed on August 1-3. In this example, the result for the negative control (“Neg”) is multiplied by 1.5 to determine the cutoff optical density at 450 nm (“OD₄₅₀”) for positivity (“Cut Off”); all experimental values relating to the evolution of color in the ELISA are referred to as EIA Units, as described in Example 1 above.

TABLE 1

| | | | | EIA Units | | | |
|---|---|---|---|---|---|---|---|
| | | | | | Hi | Low | Cut |
| Date | Blocker | Supplier | Lot No. | Neg | Pos | Pos | Off |
| 07/01 | BSA | Sigma | 121k1421 | 0.09 | 1.65 | 0.39 | 0.13 |
| 07/31 | BSA | Sigma | 121k1421 | 0.09 | 1.49 | 0.19 | 0.13 |
| 08/01 | BSA | Sigma | 52k1264 | <0.05 | <0.05 | <0.05 | <0.05 |
| 08/02 | BSA | Sigma | 52k1264 | <0.05 | <0.05 | <0.05 | <0.05 |
| 08/03 | BSA | Sigma | 52k1264 | <0.05 | <0.05 | <0.05 | <0.05 |
| 08/06 | BSA | Sigma | 111k13571 | 0.07 | 1.43 | 0.18 | 0.10 |
| 08/08 | BSA | Sigma | 59h1141 | 0.09 | 1.72 | 0.20 | 0.13 |
| 09/24 | BSA | Sigma | 36h1183 | 0.12 | 1.31 | 0.32 | 0.17 |
| 09/27 | BSA | Sigma | 111k13571 | 0.08 | 1.24 | 0.18 | 0.12 |

In further evaluation of the anomalous results of August 1-3, lot-to-lot variability was also noted in the effect of BSA on the assay controls. Data for these tests is provided in Table 2 below. For example, focusing on the BSA supplied by SeraCare Life Sciences, Inc. (Oceanside, Calif.), the high positive results exhibited EIA Unit values between about 3 and 2.5; in contrast, a couple of lots of the SeraCare BSA were associated with EIA Unit values of about 90% less, to about 0.2. The two lots of BSA from USB Corporation (Cleveland, Ohio) both gave high positive EIA Unit values that were within the range of the higher values of the SeraCare BSA.

TABLE 2

| | | | EIA Units | | | |
|---|---|---|---|---|---|---|
| Blocker | Supplier | Lot No. | Neg | Hi Pos | Low Pos | Cut Off |
| BSA | SeraCare | 01302011 | 0.11 | 2.95 | 0.34 | 0.20 |
| BSA | SeraCare | 01301021 | 0.07 | 0.20 | 0.09 | 0.10 |
| BSA | SeraCare | 01302005 | 0.07 | 0.18 | 0.09 | 0.10 |
| BSA | SeraCare | 01402005 | 0.08 | 2.67 | 0.03 | 0.12 |
| BSA | SeraCare | 01402006 | 0.08 | 2.66 | 0.02 | 0.11 |
| BSA | SeraCare | 01402001 | 0.09 | 2.46 | 0.02 | 0.13 |
| BSA | USB | 109691 | 0.10 | 3.01 | 0.03 | 0.15 |
| BSA | USB | L1083006M | 0.09 | 2.74 | 0.02 | 0.14 |

Two additional disadvantages were identified with using BSA as blocking agent. First, plates blocked with BSA exhibited a shelf life of only about eight weeks, which is consistent with proteolytic action due to plausible impurities in the BSA preparation, whereas introduction of a commercially-prepared solution of bovine protein in PBS at neutral pH, used as a blocking agent, namely StabilCoat® (SurModics, Inc., Eden Prairie, Minn.), resulted in a shelf-life of at least 52 weeks, as shown in the data recorded in Table 3. The identity of the bovine protein indicated as contained in the StabilCoat® product is not publicly known as the product is proprietary; it may be BSA, but having a likely low concentration of proteolytic impurities.

TABLE 3

| | BSA (SeraCare) | | | StabilCoat ® | | |
|---|---|---|---|---|---|---|
| | EIA Units | | | EIA Units | | |
| Week | Hi Pos | Low Pos | Neg | Hi Pos | Low Pos | Neg |
| 0 | 1.19 | 0.18 | 0.06 | 1.32 | 0.18 | 0.07 |
| 4 | 1.07 | 0.21 | 0.06 | 1.23 | 0.22 | 0.06 |
| 8 | 1.15 | 0.18 | 0.07 | 1.54 | 0.20 | 0.08 |
| 12 | 0.81 | 0.14 | 0.07 | 1.42 | 0.18 | 0.07 |
| 16 | 0.82 | 0.16 | 0.07 | 1.53 | 0.25 | 0.08 |
| 25 | 0.80 | 0.14 | 0.07 | 1.62 | 0.24 | 0.08 |
| 34 | 0.66 | 0.11 | 0.06 | 1.83 | 0.18 | 0.06 |
| 43 | 0.59 | 0.10 | 0.06 | 1.45 | 0.22 | 0.06 |
| 52 | 0.48 | 0.09 | 0.05 | 1.62 | 0.17 | 0.05 |

Second, precision for the assay control was lower using BSA compared to using a commercially-available blocker sold for immunoassays, such as, for example StartingBlock™ blocking buffer (Pierce Biotechnology, Inc., Rockford, Ill.) or StabilCoat® blocking buffer (Surmodics, Inc., Eden Prairie, Minn.), the precise recipes of which are proprietary. The StartingBlock™ buffer contains a proprietary protein preparation in a PBS or TBS buffer; the StabilCoat® buffer, as noted above, contains a bovine protein of unknown identity. In this analysis, the coefficient of variation (“Coeff Var,” obtained by dividing the standard deviation by the mean of a statistical sample) was about 0.23% in BSA plates compared to about 0.10% using the StabilCoat or StartingBlock, thereby demonstrating the increased precision of the assay.

TABLE 4

| Blocker | Parameter | Neg | Hi Pos | Low Pos | Cut Off |
|---|---|---|---|---|---|
| BSA | SD | 0.02 | 0.48 | 0.06 | 0.10 |
| | Mean^{⋄} | 0.07 | 2.10 | 0.23 | |
| | Coeff Var | 0.24 | 0.23 | 0.24 | |
| StabilCoat ® | SD | 0.01 | 0.22 | 0.03 | 0.10 |
| | Mean^{⋄} | 0.07 | 2.72 | 0.22 | |
| | Coeff Var | 0.10 | 0.08 | 0.13 | |
| StartingBlock ™ | SD | 0.01 | 0.38 | 0.01 | 0.10 |
| | Mean^{⋄} | 0.07 | 2.57 | 0.22 | |
| | Coeff Var | 0.03 | 0.15 | 0.06 | |

^{⋄}Expressed in EIA Units, as defined in Example 1.

Accordingly, selection of a BSA having a lower coefficient of variation and/or a proteolysis inhibitor, or the complete removal of BSA from the ELISA diagnostic protocol by use of a non-BSA blocking and stabilizing compound, not only reduces the likelihood of a false negative, increases the shelf-life of the prepared plates set up for the ELISA, but may also desirably reduce the incidence of false positive results caused by antibodies to BSA. For example, a blocking agent may include a low level of proteolytic activity via a greater purity grade of included products, especially the source of BSA, which is known to include protease as an impurity in common commercial preparations thereof. A proteolysis inhibitor may also be used in the blocking agent formulation, which may include BSA. In particular, the StabilCoat blocking buffer (containing either a more refined bovine protein having low level of detrimental impurities and/or a proteolysis inhibitor therein) was used to retest 17 specimens from proven histoplasmosis cases that tested falsely as negative using the standard protocol that used BSA as blocker. Six of the 17 false negative specimens by the prior ELISA were positive in the improved assay hereof.

### Example 3

This example sets forth a comparative assessment of various commercially-available blocking agents for use as a plate blbcker in an ELISA test for Histoplasma antigen detection.

Five different commercially-available blocking agents were tested as plate blockers in the Histoplasma ELISA set forth in Example 1. The five blocking agents tested were Blocker Casein, Sea Block, Starting Block™, Super Block®, and StabilCoat® (SurModics; the prior four agents were all sourced from Pierce Biotechnology, Inc.). The results are presented in the following table:

TABLE 5

| Blocker | Neg | Hi Pos | Low Pos | GARA | Cut Off |
|---|---|---|---|---|---|
| Blocker Casein | 0.092 | 2.845 | 0.239 | 2.148 | 0.138 |
| SeaBlock | 0.088 | 1.127 | 0.120 | 3.762 | 0.132 |
| Starting Block ™ | 0.076 | 2.750 | 0.214 | 1.670 | 0.114 |
| Super Block ® | 0.099 | 2.704 | 0.218 | 1.431 | 0.150 |
| StabilCoat ® | 0.074 | 2.727 | 0.192 | 1.678 | 0.111 |

The Blocker Casein and SeaBlock products were both excluded because of the higher result in the false-positive control specimen that contained goat anti-rabbit antibodies (“GARA”; fifth column). SeaBlock was also excluded because of the significantly reduced results in the high positive (“Hi Pos”) and low positive (“Low Pos”) Histoplasma controls. Comparable results were observed with Starting Block™, Super Block®, and StabilCoat®. Among these, Starting Block™ was selected for further analysis because it is known to contain only plant proteins, thereby reducing the likelihood of false-positive results caused by antibodies to animal proteins that may be contained in a physiological sample to be tested, or the rabbit antibodies used in the antigen assay as detector antibody.

### Example 4

This example illustrates an approach for minimizing false positive results from the ELISA assay set forth in Example 1 hereof.

A preparation of polyclonal antibodies that were raised in a rabbit inoculated with Histoplasma antigen in accordance with well-established procedures was treated with the enzyme papain or pepsin, which cleaves IgG molecules to generate F_{c} and F(ab)′₂ fragments, respectively. Accordingly, a preparation of the F(ab)′₂ fragment was generated, again using procedures well-known in the art. See Cerottini, “An antigen-binding capacity test for human immunoglobulin G (IgG) fragments.” J. Immunol. 101:433-438 (1968).

The ELISA assay as set forth at Example 1 was altered with respect to the antibody preparation used. Instead of using a standard polyclonal antibody that recognizes Histoplasma antigen, the aforementioned F(ab)′₂ fragment was employed, and tested for how it impacted sensitivity and error when used as the capture antibody or the detector antibody or both.

Additionally, to guard against interference caused by the possible inclusion of antibodies to rabbit IgG (whether the F(ab)′₂ fragment or whole IgG) in a serum sample from a patient (referred to herein as “HARA”), normal rabbit serum (“NRS”) was added to the detector antibody preparation. In the experiments set forth in this example, the detector antibody was a preparation of biotinylated IgG or F(ab)′₂ anti-Histoplasma antibody in the presence of excess NRS. (The role of the NRS is to absorb out any HARA, i.e., antibodies to the F(ab)′₂ that might be present.)

The following two tables set forth data from separate experiments where the capture antibody used in the procedure of Example 1 was a 25 μg/ml solution of IgG or F(ab)′₂ anti-Histoplasma antibody from which the antibodies were separately adsorbed to a surface. And in other separate experiments, the detector antibody was a biotinylated IgG or F(ab)′₂ anti-Histoplasma antibody. In each of the experiments, only the capture antibody or the detector antibody was replaced with the F(ab)′₂ fragment, not both.

TABLE 6A

| Capture Ab | Neg | Hi Pos | Low Pos | Cut Off |
|---|---|---|---|---|
| IgG 25 μg/ml | 0.06 | 2.19 | 0.29 | 0.09 |
| F(ab)′₂ 25 | 0.06 | 1.06 | 0.16 | 0.09 |
| μg/ml | | | | |

TABLE 6B

| Detector Ab | Neg | Hi Pos | Low Pos | Cut Off |
|---|---|---|---|---|
| IgG | 0.05 | 1.94 | 0.14 | 0.08 |
| F(ab)′₂ | 0.06 | 2.90 | 0.16 | 0.09 |

Results in the HP and LP controls were lower using F(ab)′₂as capture antibody compared to IgG, whereas the opposite findings occurred using F(ab)′₂ as biotinylated detector antibody. Subsequent research focused on use of F(ab)′₂ as the biotinylated detector antibody.

To evaluate the ability of biotinylated F(ab)′₂ (“B-F(ab)′₂”) used as the detector antibody to reduce false-positive results, it was compared to biotinylated IgG (“B-IgG”) in an experiment evaluating the high and low positive Histoplasma antigen controls, as well as a goat anti-rabbit antibody (“GARA”) control and a false-positive specimen exhibiting HARA activity from a patient who did not have histoplasmosis. As an additional measure to reduce HARA interference, the biotinylated F(ab)′₂ was studied in the presence or absence of normal rabbit IgG (“NR-IgG”). In all other respects, the procedure set forth in Example 1 was employed. The data are presented in the following table.

TABLE 7

| Detector | | | | | HARA | |
|---|---|---|---|---|---|---|
| Antibody | Neg | Hi Pos | Low Pos | GARA | Patient | Cut Off |
| B-IgG | 0.06 | 2.25 | 0.18 | 0.82 | 3.74 | 0.09 |
| B-F(ab)′₂ | 0.09 | 3.24 | 0.26 | 0.56 | 1.10 | 0.12 |
| B-F(ab)′₂ + | 0.08 | 3.20 | 0.22 | 0.20 | 0.11 | 0.12 |
| NR-IgG | | | | | | |

Although replacement of B-IgG with B-F(ab)′₂ alone significantly reduced the GARA control and false positive HARA patient sample, the greatest reduction occurred when B-F(ab)′₂ was used in the presence of NR-IgG. As noted above, the results of the high positive (“Hi Pos”) and low positive (“Low Pos”) Histoplasma antigen control specimen were higher in the B-F(ab)′₂ protocols, with or without NR-IgG.

Subsequent experiments determined if normal rabbit serum (“NRS”) could be used in place of NR-IgG. Again using the protocol set forth in Example 1 in all other respects, nearly identical results were found using 10% NRS or 10 μl/ml NR-IgG in excess, as seen in the following data:

TABLE 8

| Detector | | | | | |
|---|---|---|---|---|---|
| Antibody | Neg | Hi Pos | Low Pos | GARA | Cut Off |
| B-F(ab)′₂) + | 0.13 | 2.91 | 0.28 | 0.06 | 0.20 |
| NR-IgG | | | | | |
| B-F(ab)′₂ + | 0.10 | 2.90 | 0.28 | 0.05 | 0.15 |
| NRS | | | | | |

To screen for animal to animal variability in the ability of NRS to reduce interference from GARA activity, serum specimens were separately obtained from a large group of rabbits and evaluated as the diluent for B-F(ab)′₂. In all other respects, the experiment was as stated at Example 1.

TABLE 9

| Rabbit ID | Neg | Hi Pos | Low Pos | GARA | Cut Off |
|---|---|---|---|---|---|
| 1273 | 0.06 | 2.14 | 0.26 | 2.72 | 0.09 |
| 1275 | 0.05 | 2.32 | 0.29 | 0.11 | 0.08 |
| 1662 | 0.08 | 2.70 | 0.31 | 0.11 | 0.12 |
| 1663 | 0.04 | 2.53 | 0.26 | 0.25 | 0.08 |
| 1664 | 0.03 | 2.58 | 0.26 | 0.20 | 0.06 |
| 1666 | 0.04 | 2.60 | 0.29 | 0.21 | 0.08 |
| 1667 | 0.05 | 2.41 | 0.24 | 2.07 | 0.10 |
| 1670 | 0.03 | 2.34 | 0.22 | 0.17 | 0.06 |
| 1673 | 0.05 | 2.47 | 0.32 | 2.74 | 0.08 |
| 1676 | 0.05 | 2.35 | 0.29 | 0.13 | 0.08 |
| 1683 | 0.04 | 2.25 | 0.30 | 2.25 | 0.06 |
| 1685 | 0.04 | 2.34 | 0.24 | 0.16 | 0.08 |
| 1686 | 0.04 | 2.28 | 0.22 | 0.13 | 0.08 |
| 1687 | 0.07 | 2.39 | 0.28 | 0.12 | 0.11 |
| 1688 | 0.04 | 2.30 | 0.24 | 0.17 | 0.08 |
| 1689 | 0.04 | 2.30 | 0.23 | 0.06 | 0.08 |
| 2839 | 0.06 | 2.30 | 0.28 | 0.26 | 0.09 |
| 2843 | 0.06 | 2.41 | 0.29 | 0.13 | 0.09 |
| 2844 | 0.07 | 1.92 | 0.21 | 0.12 | 0.11 |
| 2845 | 0.08 | 2.22 | 0.26 | 2.79 | 0.12 |
| 9194 | 0.05 | 2.32 | 0.31 | 0.12 | 0.08 |
| 9196 | 0.05 | 2.45 | 0.34 | 0.13 | 0.08 |
| 9200 | 0.05 | 2.47 | 0.34 | 0.30 | 0.08 |
| 9201 | 0.08 | 1.79 | 0.24 | 2.65 | 0.12 |
| 9203 | 0.04 | 2.04 | 0.25 | 0.15 | 0.08 |
| 9206 | 0.07 | 1.04 | 0.28 | 0.28 | 0.11 |

Remarkably, great variability was noted in the ability of serum from different rabbits to reduce interference in the ELISA test due to inclusion of GARA. The 26 rabbits tested, 6 failed to reduce GARA (i.e., Rabbit ID Nos. 1273, 1667, 1673, 1683, 2845, and 9201), and three reduced detection of the high positive control (i.e., Rabbit ID Nos. 2844, 9201 and 9206). Thus, rabbits to be used for production of NRS for use in the assay are preferably screened to avoid using normal rabbit serum that cannot absorb out HARA.

Results for patients for whom both urine and serum specimens were available and tested together were compared for one month using the old assay (as set forth at Example 1 hereof) and the new assay set forth in this example. The percentage of positive results increased from 7.0% (51/725) in the old assay versus 11.6% (108/931) in the new assay. Conversely, false-positive results declined from 7.3% (4/55) of all positive results in the old assay versus 2.7% (3/111) in the new assay.

### Example 5

This example sets forth one particular ELISA protocol, recited in a standard operating procedure format. This protocol is predicated on the improvements understood by means of the examples set forth herein above, and shows that the various improvements that are individually assessed above do indeed reduce the incidence of false positive and false. negative results.

Phvsiological specimen handling, processing, and storage. Serum, urine, CSF, BALF and other sterile body fluids are all acceptable, so long as no assay-interfering-concentration of a chelating agent (EDTA or EGTA, for example) is present. Minimum volume is 0.5 ml for all specimens, however 2 ml is preferred. If a specimen will not be tested within 24 hours of receipt, it is stored at 4° C. until testing occurs.

Serum is separated from the clot. No refrigeration, freezing or specimen preservation is required. Whole blood is centrifuged under standard conditions to pellet cells and separate same from the serum. Aliquots of serum are transferred with a standard transfer pipet into reaction wells. Other types of specimens are tested without further processing, unless particulate matter in the specimen interferes with pipetting, in which case the specimen is centrifuged and the supernatant pipetted for use in the antigen test.

Test Specimens. As a test of the new protocol, specimens from patients with histoplasmosis and controls with false-positive results known to have been caused by HARA were compared in the protocol set forth in this example as compared to the old assay using BSA as the plate blocking reagent and IgG as the capture and biotinylated detector antibody. The histoplasmosis case specimens included 23 specimens that were positive and 33 that were falsely negative in the assay set forth at Example 1. The control group consisted of 21 specimens from organ transplant patients who had received treatment with anti-rabbit antibodies and exhibited false-positive results caused by HARA activity.

Reagents.

(1) Blocking agents. StartingBlock® TBS (Catalog #37542, Pierce, Rockford, Ill.) and StartingBlock® PBS (Catalog #37538, Pierce, Rockford, Ill.) were purchased, both of which are protein-based in either a tris-buffered or phosphate-buffered saline. PBS blocking solution is used undiluted from the bottle to coat the plates; coated plates are not used after 90 days; TBS assay diluent is used undiluted from the bottle, and stored at 4° C.

(2) Biotin-coniugated anti-Histoplasma Antigen rabbit F(ab)₂ & IgG. Rabbit polyclonal, Protein A-purified IgG that was specific for Histoplasma antigen was supplied to Strategic Biosolutions (Newark, Del.) for accessing its service for digesting the antibody with pepsin to create a F(ab)′₂ fragment. The F(ab)′₂ fragment was then provided to Vector Laboratories (Burlingame, Calif.) for its service for conjugating the F(ab)′₂ to biotin. A working dilution of the F(ab)′₂ is made by diluting in StartingBlock TBS, 1:3633 from the stock received from Vector Laboratories, to a final concentration of 200 to 300 ng/ml as determined by titration. In this way, we optimized discrimination of positive and negative controls, and maintained the negative control optical density at 450/630 nm below 0.10.

(3) Normal rabbit serum (NRS). Serum from Flemish Giant rabbits housed at Lampire Biologicals (Pipersville, Pa.) is collected twice monthly and pooled to form NRS used in the daily clinical testing. NRS is aliquoted and stored at −80° C. Enough for one week's testing is thawed each Monday morning and stored at 4° C., and combined with the diluted biotinylated F(ab)′₂ to a final concentration of 5%.

(4) Streptavidin-HRP Conjugate. Streptavidin-HRP is purchased lyophilized from Roche (Catalog #1089153, Streptavidin-POD); reconstituted in 1.0 ml of autoclaved ultrafiltration (UF) filtered water. Reconstituted streptavidin-HRP is replaced after no more than 6 months and is stored at 4° C. A working dilution is made by diluting in StartingBlock TBS, 1:50,000 from the stock.

(5) Color generation system. TMB Microwell Peroxidase Substrate System (BioFX TMB1) was purchased from KPL (Gaithersburg, Md.). This is a single component system and must be at room temperature prior to use. The chromogenic substrate needs to be protected from light to avoid degradation. Substrate system is stored refrigerated at 4° C.

(6) 0.01 M Tris-HCI Buffer. To prepare a 10× stock, combine: (i) 12.1 g Tris Base (Sigma Chemicals, St. Louis; Catalog #T-8524); (ii) add 900 ml UF filtered water to dissolve; (iii) pH to 7.0 with concentrated HCI; and (iv) bring final volume to 1000 ml with UF filtered water. Store at 4° C. Stock is replaced after no more than 6 months. Before use, dilute to 1× with UF filtered water. Store refrigerated.

(7) 0.1 M Tris-Saline Buffer. To prepare 10× stock, combine: (i) 121.1 g Tris Base (Sigma, Catalog #T-8524); (ii) 85.0 g NaCl (Sigma, Catalog #F-9625); (iii) add 900 ml UF filtered water to dissolve; (iv) pH to 8.0 with concentrated HCl; and (v) bring final volume to 1000 ml with UF filtered water. Store at 4° C. Stock is replaced after no longer than 6 months. Before use, dilute to 1× with UF filtered water. Store refrigerated.

(8) PBS EIA Wash. To prepare 10× stock, combine: (i) 80.0 g NaCl (Sigma, Catalog #F-9625); (ii) 2.0 g KCl (Fisher Chemical, Catalog #P217); (iii) 14.4 g Na₂HPO₄ (Sigma, Catalog #S-0876); (iv) 2.4g KH₂PO₄ (Sigma, Catalog #P-53779); (v) add 900 ml UF filtered water to dissolve; (vi) pH to 7.2 with NaOH; (vii) add 5.0 ml Tween® 20 (Sigma, Catalog #P-1379); and (viii) bring final volume to 1000 ml with UF filtered water. Store at room temperature. Stock is replaced after no longer than 6 months. Dilute to 1× with UF filtered water. Filtering is not necessary; use wash only on the day it is diluted to 1×.

(9) 2.0 N Sulfuric Acid (LabChem Inc., Catalog #LC25790-1).

(10) Positive Controls. The positive controls consist of a High and a Low Positive of concentrated known Histoplasma antigen positive urine (5.3× stock). High Positive Control Urine is diluted 1:20 from 5.3× stock−5.0 ml concentrated urine+95.0 ml 0.1 M Tris. Low Positive Control Urine is diluted 1:2000, i.e., 1.0 ml High Positive Control urine+99.0 ml 0.1 M Tris. For quality control testing, new positive controls are tested in tandem with existing controls. New lots have at most 20% mean difference from the existing controls to be accepted. Current controls are stored in the refrigerator. Aliquots of accepted controls not in use are stored frozen.

(11) Negative Controls & Cut-Off Calibrators. 0.1 M Tris-Saline pH 8.0 can serve as both the negative control and cutoff calibrators. For quality control testing, the new Tris solution is tested in tandem with the existing solution. New lots have at most 20% mean difference from the existing lot to be accepted. Currently used negative controls and cut-off calibrators are stored in the refrigerator. Aliquots of accepted controls/calibrators not in use are stored frozen.

ELISA procedure. Remove appropriate number of precoated plates from the sealed bags for the number of specimens to be tested. All wells not being used should be removed from the plate and returned with a desiccant to storage.

Add 100 μl/well of the control or specimen to be tested. All samples are tested in the following order on each plate: Cutoff Calibrator, cutoff calibrator, high positive control, low positive control, negative control, patient samples. Patient samples are loaded in alphabetical order according to the work list.

Seal each plate and incubate at 37° C. for 1 hour. Then wash the plate five times with freshly prepared 1× PBS EIA wash prepared daily. Next, apply a priming wash of purified water into each well, and remove. Then add 100 μl/well of a 1:3633 dilution of biotin-conjugated anti-Histoplasma rabbit F(ab)₂ with 5% NRS in StartingBlock TBS®. Reseal each plate and incubate at 37° C. for 1 hour.

Wash the plate five times with 1X PBS EIA wash as above. Add 100 μl/well of a 1:50,000 dilution of HRP-labeled streptavidin in StartingBlock® TBS. Prepare a 1:1000 dilution first and from this make a 1:50 dilution to end up with a final 1:50,000 dilution factor. Reseal each plate and incubate at 37° C. for 1 hour. Wash the plate five times with 1× PBS EIA wash as described above.

Add 100 μl/well of room temperature TMB1 Peroxidase Substrate (Kirkegaard & Perry Laboratories, Gaithersburg, Md.). Develop the plate in the dark for 8 minutes at room temperature. Stop the reaction by adding 100 μl/well of 2.0 N sulfuric acid. Color development is measured by reading the optical density on the Tecan EIA Plate reader at 450 nm then 630 nm wavelengths.

Data. Of the 33 falsely-negative specimens from patients with histoplasmosis, 24 were positive in the new B-F(ab)′₂ assay. Furthermore, only one of the 21 false-positive HARA specimens were positive in the new B-F(ab)′₂ assay.

Sensitivity in false negative cases improved 73% and specifically in false positive cases improved about 95% using the new method. Accordingly, false negative results in individuals with disseminated histoplasmosis are expected to be less than 10% and in acute pulmonary histoplasmosis less than 20%. False positive results are expected to be rare in the new assay.

### Example 6

This example presents a Histoplasma antigen assay comprising a quantitative reporting step that can be used to monitor the effect of treatment of histoplasmosis. Most immunoassay test results typically express the antigen levels semiquantitatively by comparison to a cutoff derived by multiplication of the negative control by a factor ranging from 1.5-2.0. However, due to day-to-day variability in the antigen assay, this semiquantitative approach to reporting test results required testing of the prior sample along with the current sample in the same assay to assess the change in antigen during treatment. In contrast, the quantitative reporting method used in this example permits a determination of antigen concentration by comparison to a calibration curve, so as not to require repeated testing of the prior sample with the current sample.

In this example, an Histoplasma antigen ELISA test was performed to detect a yeast cell wall galactomannan in the Histoplasma antigen. Evidence for this includes the following: stability at 100° C., resistant to proteases, susceptible to glycosidases and sodium periodate (See, Wheat, L. J., R. B. Kohler, and R. P. Tewari, “Diagnosis of disseminated histoplasmosis by detection of Histoplasma capsulatum antigen in serum and urine specimens,” N. Engl. J. Med. 314:83-88 (1986), incorporated herein by reference in its entirety) and affinity to concanavalin A. Thus, purified Histoplasma yeast galactomannan was selected as a calibration standard for antigen detection.

Preparation of Purified Histoplasma galactomannan

Histoplasma capsulatum yeast, obtained from a clinical patient isolate, was grown in 12×1.0 L flasks containing 300.0 ml of brain heart infusion broth incubated at 37° C. on a gyratory shaker (New Brunswick Scientific Co., Inc., New Brunswick, N.J.) for 72 h. The yeast cells were then harvested by centrifugation at 4420×g for 10 min at 4° C. (Beckman J2-21 M/E centrifuge). The supernatant was discarded and the pelleted cells were resuspended in 250 ml 0.25 M NaOH. The suspension was mixed for 16-18 h at 4° C. Afterwards, the suspension was centrifuged at 921× g for 10 min at 4° C.(B. Braun Biotech model sigma 6k 10 centrifuge, Allentown, Pa.). The supernatant was decanted and combined with three volumes of absolute ethanol. The solution was stirred for 16-18 h. at 4° C. and afterwards centrifuged at 3685×g for 10 min at 4° C. The supernatant was discarded and the pellet was solubilized with deionized water and neutralized to a pH of 7.2 using 17.4 M glacial acetic acid. The neutralized extract was spun down at 3685×g for 10 min at 4° C. and the supernatant was dialyzed extensively against deionized water using 1000 MWCO tubing. The yeast extract was purified by immunoaffinity chromatography using concanavalin A, molecular sieve chromatography using CL-6B sepharose, and ion exchange chromatography using DEAE cellulose.

A 2.5×40.0 cm column containing 105.0 ml bed volume of glutaraldehyde treated. Con A sepharose was prepared according to instructions (Pharmacia Biotech, Inc., Piscataway, N.J.). The lyophilized extract was reconstituted in 24 ml of Con A buffer (0.2 M tris, 0.5 M NaCl, 1.0 mM MnCl₂, 1.0 mM CaCl₂) and was loaded on the column. The loaded extract was allowed to react for 1.0 h and afterwards the column was washed with 2.0 L of Con A buffer at a flow rate of 0.25 ml/min as controlled by a peristaltic pump.(Bio Rad, Hercules, Calif.) Washing was monitored in the Histoplasma capsulatum immunoassay and was halted when the antigen reactivity stopped decreasing and was considered stable. The column was eluted using 0.5 M α-methyl-D-mannopyranoside in Con A buffer. Fractions were monitored at A₂₁₃, A₂₈₀ and in the previously described immunoassay. Based on these results, fractions with an A₂₁₃>0 and strong reactivity in the immunoassay were considered to contain a significant amount of antigenic material and were pooled. The pooled fractions were concentrated to a volume of<100.0 ml, using a prep/scale tangential flow concentrator fitted with a prep/scale TFF1ft² 1000 MWCO cartridge and peristaltic pump(Millipore Corporation, Bedford, Mass.), reconstituted to 1.0 L with DI H₂O, and concentrated again to<100.0 ml.

A 1.6×75.0 cm column containing 126.7 ml bed volume of CL-6B sepharose was prepared according to instructions (Pharmacia Biotech, Inc., Piscataway, N.J.). The Con A purified extract was reconstituted in 6.38 ml of 0.2 M NaCl and was spun at 3685×g for 20 min to remove any insoluble particulate material. Next, 6.269 ml was loaded on the column via gravity. Elution was carried out using 0.2 M NaCl at a flow rate of 0.07 ml/min as controlled by a peristaltic pump (Bio Rad, Hercules, Calif.). Approximately 4.2 ml fractions were collected. Elution was monitored by testing the fractions at a 1/10⁵ dilution in the Histoplasma capsulatum immunoassay and reading the fractions at a 1/10 dilution at A₂₂₀ and A₂₈₀. Based on these results, fractions, which displayed significant activity in the immunoassay and A₂₂₀>0.2, were combined and dialyzed against deionized water using 1000 MWCO tubing. The dialyzed extract was frozen and

lyophilized.

A 1.6×24.5 cm column containing 49.0 ml bed volume of DEAE sepharose A-50 was prepared according to instructions (Pharmacia Biotech, Inc., Piscataway, N.J.). The Con A/CL-6B purified extract was reconstituted in 6.38 ml of 0.2 M NaCl and was spun at 3685×g for 20 min to remove any insoluble particulate material. Next, 6.269 ml was loaded on the column via gravity. Elution was carried out using 0.2 M NaCl at a flow rate of 0.07 ml/min as controlled by a peristaltic pump (Bio Rad, Hercules, Calif.). Approximately 4.2 ml fractions were collected. Elution was monitored by testing the fractions at a 1/10⁵ dilution in the Histoplasma capsulatum immunoassay and reading the fractions at a 1/10 dilution atA₂₂₀ and A₂₈₀. Based on these results, fractions, which displayed significant activity in the immunoassay and A₂₂₀>0.2, were combined and dialyzed against deionized water using 1000 MWCO tubing. The dialyzed extract was frozen and lyophilized.

Preparation of Assay Calibration Standards

Calibration standards were prepared from a pool of urine specimens containing high levels of Histoplasma antigen. Urine specimens were first screened for cross-reactivity in the Platelia Aspergillus galactomannan antigenemia assay (BioRad), and those that were positive were excluded from the calibrator pool. Multiple dilutions of the calibrator pool were prepared, and the antigen content of each calibrator was determined by comparison to known concentrations of the purified galactomannan, at concentrations ranging from 0.6 ng/ml to 39 ng/ml. Accordingly, urine calibrators were assigned ng/ml concentration values.

Determination of Antigen Concentration.

Results were classified as positive or negative by comparison of the optical density of the test specimen to that of the no antigen control specimen. Galactomannan concentration of specimens determined to be posited by comparison to the negative control was determined by comparing the optical density of the test specimen to that of the calibration curve standards, and results were expressed ng/ml. Specimens with results exceeding the cutoff for the assay but less than the lowest standard were reported as positive, less than 0.6 ng/ml, and results higher than the 39 ng/ml calibrator as ≧39 ng/ml.

Quantitation of Antigen Concentration.

An example of the standard curve usually, calibration standards prepared from the urine from patients with histoplasmosis is shown in FIG. 6A, discussed above. The standard curve was highly reproducible when determined on multiple occasions.

Results for ten representing patient tested on two occasions are shown in table 10, providing an example of the reproducibility of the quantitative method. The average difference between test 1 and test 2 was larger for results expressed semiquantitatively as antigen units (36%) than quantitatively as ng/ml (14%).

TABLE 10
Reproducibility of Antigen Level Determined
on Two Different Days Expressed As Antigen
Units or Antigen Concentration in ng/ml.

| | | | % Differ- | Antigen | | % Differ- |
|---|---|---|---|---|---|---|
| | | | ence | concentration | | ence |
| Patient | Antigen units | | [T2 − | [ng/ml] | | [T2 − |
| No. | Test 1 | Test 2 | T1/T1] | Test 1 | Test 2 | T1/T1] |
| 1 | 10.7 | 14.4 | 35% | 9.5 | 10.1 | 6% |
| 2 | 8.4 | 12.2 | 45% | 7.9 | 8.3 | 5% |
| 3 | 14.3 | 18.4 | 29% | 12.1 | 13.4 | 11% |
| 4 | 18.8 | 28.4 | 48% | 16.0 | 23.5 | 47% |
| 5 | 21.8 | 29.7 | 37% | 19.4 | 24.9 | 28% |
| 6 | 25.8 | 33.3 | 29% | 26.7 | 29.4 | 10% |
| 7 | 8.3 | 11.2 | 35% | 7.8 | 7.6 | 3% |
| 8 | 3.1 | 4.1 | 32% | 2.0 | 2.0 | 0% |
| 9 | 4.1, | 5.4 | 32% | 3.9 | 3.0 | 23% |
| 10 | 6.8 | 9.3 | 37% | 6.6 | 6.1 | 8% |

| | | |
|---|---|---|
| Average | 36% | 14% |

As a further example of the superiority of the quantitative method, results for sequential specimens from a patient with histoplasmosis are contrasted when tested together in the same day or five different days (FIGS. 7A-7B, as discussed above). Results from the same versus different days agreed more closely in the quantitative assay, expressed as ng/ml, than in the semi quantitative assay, expressed as EIA units. FIGS. 7A and 7B show an example of testing sequential specimens from a single patient in the same assay (same day) or in five different assays (different days), as discussed above.

### Example 7 Cross-reactivity

This example describes the ability of the Histoplasma immunoassay detect (i.e., cross-react with) other antigens. Over 70% of specimens from patients with blastomycosis, paracoccidioidomycosis, penicilliosis marneffei and African histoplasmosis, caused by H. capsulatum var. duboisii were positive in the Histoplasma antigen assay, at levels similar to those in histoplasmosis (as described in Wheat J, Wheat H, Connolly P et al. Cross-reactivity in Histoplasma capsulatum variety capsulatum antigen assays of urine samples from patients with endemic mycoses. Clin Infect Dis 1997; 24:1169-71). Galactomannan in the cell wall of H. capsulatum is known to cross react with antibodies to Blastomyces dermatitidis and Paracoccidioides brasiliensis (as described in Azuma I, Kanetsuna F, Tanaka Y, Yamamura Y, and Carbonell L M. Chemical and immunological properties of galactomannans obtained from: Histoplasma duboisii, Histoplasma capsulatum, Paracoccidioides brasiliensis and Blasomvces dermatitidis. Mycopatholog Mycolog Appl 1974; 54:111-25.).

Cross reactions in urine from patients with coccidioidomycosis or aspergillosis were not observed. Combining results from several reports evaluating cross-reactivity in the Histoplasma or Blastomyces (as described in Durkin M, Witt J, LeMonte A, Wheat B, and Connolly P. Antigen Assay with the Potential To Aid in Diagnosis of Blastomycosis. J Clin Microbiol 2004; 42:4873-5) antigen assay, positive results were noted in one of 28 (3.6%) patients with coccidioidomycosis (as described in Garringer T O, Wheat L J, and Brizendine E J. Comparison of an established antibody sandwich method with an inhibition method of Histoplasma capsulatum antigen detection. J Clin Microbiol 2000; 38:2909-13; Durkin M M, Connolly P A, and Wheat L J. Comparison of radioimmunoassay and enzyme-linked immunoassay methods for detection of Histoplasma capsulatum var. capsulatum antigen. J Clin Microbiol 1997; 35:2252-5; Wheat J, Wheat H, Connolly P et al. Cross-reactivity in Histoplasma capsulatum variety capsulatum antigen assays of urine samples from patients with endemic mycoses. Clin Infect Dis 1997; 24:1169-71; Wheat L J, Kohler R B, Tewari R P, Garten M, and French M L. Significance of Histoplasma antigen in the cerebrospinal fluid of patients with meningitis. Arch Intern Med 1989; 149:302-4) and one of 88 (1.1%) with aspergillosis (as described in Durkin M, Witt J, LeMonte A, Wheat B, and Connolly P. Antigen Assay with the Potential To Aid in Diagnosis of Blastomycosis. J Clin Microbiol 2004; 42:4873-5). Furthermore, the positive results in those two cases were weak (<2 units).

Galactomannan purified from Coccidioides immitis is believed to be detected in the Histoplasma antigen assay described with respect to the third embodiment above. Positive results were also observed in body fluids of patients with coccidioidomycosis, as discussed with respect to FIG. 5. Referring again to FIG. 5, antigen was detected in the urine from 11 of 19 patients (58%), including patients infected with C. posadasii and C. immitis. The coccidioidomycosis cases were classified as acute (<30 days of symptoms) or chronic (>30 days of symptoms). Antigenuria was not detected in the five chronic cases, while it was detected in the bronchoalveolar lavage fluid in one. Antigen was detected in the urine of 11 of the 14 acute cases (79%), and in a 12^{th} patient (86%) following tenfold concentration of the urine. Cross-reactive antigen detection in the Histoplasmosa antigen assay was identified from patients with blastomycosis and coccidioidomycosis. Each data point represents a single patient, with results expressed in atigen (EAI) units. Results above 1.0 antigen units are positive. The reason for low cross-reactivity in specimens from patients with coccidioidomycosis in the original Histoplasma antigen assay of Example 1 and high cross-reactivity in the assay of the third embodiment above may include greater sensitivity of the third embodiment assay (e.g., made possible by use of more effective blocking agents and F(ab′)₂ detector antibodies) and/or a broader antigenic recognition of the antibodies used in the assay according to the third embodiment (e.g., made possible by modifications to the immunization procedure).

Positive results in the Histoplasma antigen assay are rare in specimens from patients with aspergillosis. Only one of 88 (1%) specimens containing Aspergillus galactomannan was positive in Blastomyces antigen assay, and that specimen demonstrated very low-level cross-reactivity (<2 units) [4]. Also, the Aspergillus galactomannan standard used in the Aspergillus antigenemia assay was negative in the Blastomyces antigen assay. More recently we have demonstrated that serum (N=20) and bronchoalveolar lavage specimens (N=17) containing Aspergillus galactomannan were negative in Histoplasma antigen assay, supporting our findings in the Blastomyces assay.

### Example 8

### Kit for Improved Immunoassay and Associated Immunoassay Method

This example describes a kit for performing an improved immunoassay to detect a glycoprotein antigen circulating in the blood and excreted in the urine of patients with histoplasmosis. The kit can be used to perform a quantitative Histoplasma antigen EIA useful to diagnose histoplasmosis, monitor the response to therapy, and to diagnose relapse. Immunoglobulins with specific activity directed to Histoplasma antigen are employed to detect this antigen in patient samples. Purified immunoglobulin is bound to the surface of a microtiter plate. Histoplasma antigen in patient specimens will bind to the immunoglobulin and subsequently is then detected with a biotin-conjugated immunoglobulin digested to F(ab)′₂ specific to Histoplasma antigen followed by streptavidin-HRP and TMB substrate.

The kit preferably comprises the following components and reagents:
Prepared Microtiter Plates Coated with anti-Histoplasma Ag rabbit IgG and Blocked with blocking agent having a coefficient of variability of less than about 0.2%, preferably substantially free of BSA Starting Block-TBS (Pierce, Rockford, Ill.);
Conjugate Diluent, Blocking agent having a coefficient of variability of less than about 0.2%, preferably substantially free of BSA (e.g., Starting Block-TBS (Pierce, Rockford, Ill.) used undiluted from the bottle for both the biotin and streptavidin conjugates)
Screened Normal Rabbit Serum screened for desirably reducing interference with GARA, as described with respect to the second embodiment above;
Biotin-conjugated anti-Histoplasma Ag rabbit IgG detector antibody digested to F(ab)′₂: A working dilution can be made by diluting (for example, at 1:7280) in the conjugate diluent containing 5% normal rabbit serum screened for desirably reducing interference with GARA, as described above;
Streptavidin-HRP Conjugate: A working dilution is made by diluting 1:50,000 in the conjugate diluent;
TMB Substrate (e.g., a single component system);
EIA Wash Tablets (e.g., dissolve one tablet in 1 L of 18 MΩ lab quality water; wash is only good on the day it is prepared);
2.0 N Sulfuric Acid (Stop Solution);
Positive Controls (including High Positive Control and Low Positive Control);
Negative Control and Calibrator (Two negative controls may be supplied in the kit and used in each assay run as the cutoff calibrators; an additional negative control may also be used); and
Quantitative Curve Standards (Nine standards supplied in the kit are run in each assay to plot the standard curve; the standards can include: 39, 28, 19, 14, 10, 6.0, 3.4, 1.7, and 0.6 ng/ml concentrations).

The kit may be used in combination with the following supplies and equipment:
ELISA plate reader;
Water purification system;
Vortex;
Laboratory Refrigerator;
37° C. Incubator;
Immuno plate washer with vacuum pressure station;
Single and Multichannel Pipettors with disposable tips;
Non sterile gloves;
Face shield or goggles;
Plate Sealer (provided in kit); and
A computer with software capable of conducting a 4 parameter curve analysis

Preferably, the assay controls meet the following criteria:
For the assay to be acceptable the controls must meet the following criteria:
   Mean negative calibrator controls must have an OD₄₅₀-OD₆₂₀<0.100.
   Negative control must be less than the calculated assay cutoff.
   Low positive control must be 4.4 ng/ml±1.0 ng/ml.
   High positive control must be≧10 ng/ml.
Do not use reagents beyond the expiration date
Any positive specimen will be repeated to confirm the positive result.

The kit may be used in accordance with the following procedure:
1. All specimens are handled following universal precautions.
2. Remove appropriate number of precoated plates/wells from the refrigerator. Plates have removable strips of wells. All wells not being used should be removed from the plate and returned to storage pouch and placed back in the refrigerator. Do not remove the desiccant pouch from the plate storage bag.
3. Allow components to come to room temperature (approximately 20 minutes).
4. Add 100 μl/well of the control or specimen to be tested. All samples are tested in the following order on each plate: Negative calibrator control 1, negative calibrator control 2, high positive control, low positive control, negative control, quantitative curve standards, patient samples.
5. Seal each plate and incubate at 37° C. for 1 hour.
6. Wash the plate 5× with freshly prepared EIA wash using an Immuno plate washer.
7. Prepare a 5% solution of NRS in conjugate diluent, and prepare a 1:7,280 dilution of biotin-conjugate into that solution, and add 100 μl/well to the microtiter plate.
8. Reseal each plate and incubate at 37° C. for 1 hour.
9. Wash the plate 5× with EIA wash as in step 6 above.
10. Prepare a 1:50,000 dilution of HRP labeled streptavidin in the conjugate diluent by first preparing a 1:1000 dilution (1 μL in 1 mL) and from this make a 1:50 dilution for a final 1:50,000 dilution. Add 100 μl/well to the microtiter plate.
11. Reseal each plate and incubate at 37° C. for 1 hour.
12. Wash the plate 5× with EIA wash as described in step 6.
13. Add 100 μl/well of TMB Peroxidase Substrate that has been brought to room temperature. Develop the plate for 12 minutes at room temperature without a plate sealer. Do not place the plate in direct light during the development time.
14. Stop the reaction by adding 100 μl/well of 2.0 N sulfuric acid stop solution.
15. Color development is measured by reading the optical density on the EIA Plate reader at OD₄₅₀-OD₆₂₀. The plate should be read within 30 minutes of adding the stop solution.

To calculate the assay cutoff the following equation may be used: Mean OD of negative calibrator controls×Multiplier (M)=Cutoff, where M=2.0 if the mean of the negative calibrator controls is >0.050 and M=3.0 if the mean of the negative calibrator controls is ≦0.050. Preferably, the negative control is less than the cutoff.

To plot the calibration curve shown in FIG. 6B, the following 4 parameter formula was used:

$$y = min + \frac{max - min}{1 + {( x / EC 50 )}^{Hillslope}}$$

The controls are preferably selected to meet the following criteria: Low positive control that is 4.4 ng/ml±1.0 ng/ml and a High positive control must be ≧10 ng/ml. Preferably, the R² value for the line is ≧0.98.

Using the calibration curve of FIG. 6B, all patient results are preferably determined by calculating from the standard curve. Patients with results higher than the highest standard can be reported as >39 ng/ml and patients with results lower than the lowest standard, but higher than the cutoff, can be reported as positive, <0.6 ng/ml. Patients with results less than the cutoff can be reported as “none detected.” Using the calibration curve of FIG. 6B, the reportable range is <0.6->39 ng/ml. Results of none detected are negative. Results above the cutoff are positive and interpreted using the following guideline.

| Specimen Result | | Result Interpretation |
|---|---|---|
| None Detected | | Negative |
| ≦1.9 | ng/ml | Positive, borderline |
| 2.0-9.9 | ng/ml | Positive, weak |
| 10-19.9 | ng/ml | Positive, moderate |
| ≧20.0 | ng/ml | Positive, high |

Change in antigen between samples to monitor therapy is interpreted as follows:

TABLE 11

| | | Interpretation |
|---|---|---|
| Results for previous specimen: | | |
| Borderline- | High Positive | |
| Moderate | [≧20.0 ng/ml] | |
| Positive | | |
| [<20 ng/ml] | | |
| Requirement for significant change: | | |
| >4 ng/ml | >20% increase | Probable Treatment |
| increase | | Failure/Relapse |
| <4 ng/ml | <20% decrease | Possible Treatment Failure |
| decrease | | |
| >4 ng/ml | >20% decrease | Probable Treatment Response |
| decrease | | |

### Example 9

Antibodies Obtained from Vaccination of Rabbits with Single or Multiple Antigen Isolate(s)

This example describes the vaccination of rabbits with multiple Histoplasma antigens to obtain improved capture and/or detector antibodies.

Preparing Histoplasma Mould for Vaccine

A multiple-isolate rabbit vaccine was made using five Histoplasma capsulatum mould isolates from the following patients:

| Patient | Location | Sample Date |
|---|---|---|
| Patient 1 | Kansas City | Mar. 30, 2004 |
| Patient 2 | Baptist Med Center | Aug. 22, 2004 |
| Patient 3 | Univ. of Iowa | Aug. 22, 2004 |
| Patient 4 | Clarian | Jul. 06, 2004 |
| Patient 5 | Clarian | Sep. 14, 2004 |

The isolate on Patient 1 was received in the lab Mar. 30, 2004. The other isolates were grown out of urines that had been sent to MiraVista for Hc antigen. Urines that tested high positive in the antigen assay were chosen. 0.1 ml urine was streaked on Yeast Extract Phosphate Agar w/Ammonia, and mould growth was examined for the characteristic micro and macro conidia. Isolates were then grown on potato dextrose slants, and sent for Gen-Probe ID. All sent were confirmed as H. capsulatum. In order to grow a large quantity of each isolate:
1. Individual colonies were chosen from the Yeast Extract Phosphate Agar plate and were subcultured onto 2-3 Potato Dextrose Agar slants. When sufficient mould growth was seen on these slants, (approx. 2 weeks), slants were used to inoculate flasks of Potato Dextrose Broth. The procedure for “Preparation of Mould Vaccine for Rabbit Immunization” was followed (see below). 6 flasks per isolate were grown, formalin killed, washed to remove formalin and then frozen as a 30% suspension at −80° C.; and
2. When all 5 isolates had been prepared in this way, the rest of the vaccine procedure was followed. Two 50 ml conical tubes from each of the 5 isolates were thawed, blended in a Waring blender, and refrozen and sent to Lampire.

To prepare a comparative control, a control vaccine was made from a single Histoplasma capsulatum mould isolate. In order to obtain a large quantity of the isolate:
1. Minimally passaged IUCT isolate was thawed from liquid nitrogen storage and subcultured onto 2-3 Potato Dextrose Agar slants. When sufficient mould growth was seen on these slants, (approx. 2 weeks), slants were used to inoculate flasks of Potato Dextrose Broth.
2. The attached procedure was followed as it was for the isolates above in the multiple-isolate vaccine.
   Preparation of Mould Vaccine For Rabbit Immunization

A multiple-isolate vaccine was prepared from the Mould by the following steps:
1. Prepare a seed flask:
   a. Prepare about 75 ml of Potato Dextrose Broth (PDB) and place in a 250 ml flask with a cotton plug covered with foil. Autoclave on short liquid cycle and cool to room temperature.
   b. Inoculate flask with mould from a slant(s) by rinsing with fresh PDB.
   c. Flasks should be placed in to shaker at RT and set to shake at 150 rpm. If necessary, wrap a paper towel around the base of the flask for a better fit.
2. Allow the cultures to grow for at least 4 days to 1 week. Small mould balls will be growing when ready. This flask can then be used to inoculate 12 one liter flasks containing 250-300 ml sterile PDB (previously autoclaved with a cotton plug and cooled to room temperature) each with approximately 5 ml from the seed flask.
3. Place all 12 flasks into shaker incubator at RT, shaking at 150 rpm and allow to grow for at least 2 weeks.
4. Check for contamination of flasks by placing one drop on a slide (under the hood) with LPCB (lactophenol cotton blue) and examine under the microscope for a pure culture (lack of other moulds or bacterial contamination).
5. Pipette 40-50 ml of the mould culture into each of 16 centrifuge tubes (50 ml conicals) under the hood. Spin down at 2,000 rpm for 10 min. at either room temperature or 4° C.
6. Discard the supernatant (carefully decant under the hood into a container with bleach in it). Add more of the culture from the flasks to the tubes and continue to spin, decant, and add more culture until all the flasks have been spun down.
7. When all the culture broth is spun down, combine pellets into 8 conical tubes.
8. Bring volume up to about 45 ml with PBS containing 5% Formalin. Place the conicals on the rotator overnight (18 hrs) at 4° C.
9. The next day, wash the mould suspensions with sterile saline or PBS to remove all the formalin. To ensure that all the formalin has been removed, wash at least 6 times or until a formalin test strip reads less than 2.5 PPM. Suspensions can be combined into 4 conicals at this point.
10. After the formalin has been removed, place the mould in a Waring blender and dilute to a 30% suspension with sterile saline. Blend thoroughly and pour back into conicals. (Blender should be autoclaved immediately, washed, and reautoclaved for the next use)
11. Add a few drops of the final suspension from each conical to either a PDA slant or plate to check for viability. (Label each tube and slant so if one tube is contaminated or not properly killed you can properly identify it.) Check for growth in about 2 weeks (formalin killed mould should not grow).
12. Freeze at −70° C. until ready for use in a rack labeled “mould vaccine-pending viability check”. If no growth occurs on the slants or plates, the label can be updated.
   Competitive Assay Using the F(ab)′2 System to Evaluate Rabbits

Rabbit serum obtained from rabbits vaccinated with the multiple-isolate vaccine was evaluated in a competitive binding assay:
1. Prepare microtiter plates coated with anti-Histoplasma rabbit IgG and blocked with Starting Block- TBS (Pierce, Rockford, Ill.).
2. Test bleed rabbit sera and control are diluted 1/500 in SB-TBS
3. 50 ul of the diluted rabbit serum is added to wells followed by 50 ul of pos ctrl urine.
4. The plate is gently tapped to mix and placed at 37 C for one hour, then washed
5. The F(ab)′₂ biotin is prepared at 300 ng/ml in SB-TBS containing 5% NRS and added at 100 ul/well; the plate is incubated at 37 C for 1 hr, then washed
6. Streptavidin-HRP is diluted 1/50 K in SB-TBS and 100 ul/well added to the plates. The plate is incubated at 37 C for one hour, then washed
7. TMB1 is added at 100 ul/well (room temp) and allowed to develop
8. The reaction was stopped with H2SO4, and the plate was read at OD₄₅₀-OD₆₂₀
9. The % inhibition is calculated by taking the (OD of the test rabbit/OD of the normal rabbit)*100 and subtracting that result from 100 (See Table 12)

10. Rabbits exhibiting about 50% or greater inhibition are selected for further analysis

TABLE 12
Single-antigen and Multiple-Antigen Vaccine
Competitive Assay (bleed on day 220)

| | Rabbit No. | % Inhibition |
|---|---|---|
| Single- | 3981 | 62.38 |
| Antigen | 3982 | 24.60 |
| Vaccine | 3983 | 19.13 |
| | 3984 | 20.53 |
| | 3985 | 17.47 |
| Multiple- | 3986 | 23.26 |
| Antigen | 3987 | 93.03 |
| Vaccine | 3988 | 75.24 |
| | 3989 | 89.12 |
| | 3990 | 90.68 |
| | 3991 | 69.08 |
| | 3992 | 70.42 |
| | 3993 | 60.77 |
| | 3994 | 59.06 |
| | 3995 | 49.25 |
| | Pos Ctrl | 92.23 |

Capture Assay of Rabbit IgG from Antisera and Test

Rabbit serum obtained from rabbits vaccinated with the multiple-isolate vaccine was evaluated in a capture binding assay according to the following steps:
1. A small amount of test IgG is purified from serum from each rabbit being evaluated using Immunopure A Plus IgG purification kit (Pierce, Rockford, Ill.) according to manufacturer's kit instructions
2. The concentration of each IgG is determined by a reading on the spectrophotometer at OD280
3. Each test IgG along with pos control IgG is coated on microtiter plates at 12.5 ug/ml, according to the standard clinical assay plate preparation protocol
4. Negative, high and low positive controls are used to evaluate each new rabbit IgG in comparison to that being currently used in the clinical test system.

5. Results are provided in Table 13 below.

TABLE 13
Evaluation of Production Bleeds of Hc04 and Hc05 Rabbits: Jan. 26, 2006 Serum Date
(approx day 300) as Capture IgG

| | Hc05 | | | | | | | | | | |
|---|---|---|---|---|---|---|---|---|---|---|---|
| | IUCT | Hc04 | | | | | | | | | |
| | Vac | New 5 | patient | clinical | isolate | vaccine | | | | | Current |
| | 3981 | 3987 | 3988 | 3989 | 3990 | 3991 | 3992 | 3993 | 3994 | 3995 | Clinical. |
| cutoff | 0.191 | 0.045 | 0.054 | 0.038 | 0.042 | 0.072 | 0.040 | 0.046 | 0.068 | 0.025 | 0.042 |
| EU of | 0.937 | 5.415 | 4.136 | 6.860 | 7.175 | 2.292 | 3.158 | 2.703 | 0.632 | 0.613 | 6.048 |
| low pos | | | | | | | | | | | |
| EU of hi | 3.867 | 36.696 | 21.278 | 40.175 | 44.238 | 11.801 | 20.742 | 19.377 | 4.765 | 3.387 | 35.008 |
| pos | | | | | | | | | | | |

Rabbit IgG coated at 12.5 ug/ml

With the competitive assay screening analysis, 1/5 (20%) of the Hc05 rabbits which were vaccinated with vaccine IUCT as prepared for previous rabbit studies showed 50% or greater inhibition after day 200. Rabbits which were vaccinated using the mixture of 5 fresh clinical isolates had 9/10 (90%) meet criteria for selection by the competitive assay.

The one rabbit from the multiple antigen vaccine and the nine rabbits from single-antigen vaccine that met the criteria for selection from the competitive assay had IgG purified from the serum collection. Three of the nine from multiple antigen vaccine, 3987, 3989 and 3990, performed at least as well as or better than the current clinical antibody when analyzed as capture. The development of a mixed vaccine from five fresh clinical isolates is superior in comparison to past vaccines for the production of anti-Histoplasma antibodies.

What is claimed:

1. A method of detecting an antigen comprising the steps of:
a. providing an antigen binding surface;
b. contacting the antigen binding surface with an analyte comprising an antigen in a manner effective to bind the antigen to the antigen binding surface;
c. contacting the bound antigen with a detector antibody comprising a modified polyclonal rabbit anti-Histoplasma IgG antibody having a fragment antigen binding domain that is not bound to a F_{c} crystalline domain; and
d. detecting the bound antigen.

2. The method of claim 1, wherein the detector antibody comprises a F(ab)′₂ domain and does not comprise a F_{c} domain of a polyclonal rabbit anti-H. capsulatum IgG antibody.

3. The method of claim 1, wherein the step of contacting the antigen bound to the antigen binding surface with the detector antibody is performed in the presence of Normal Rabbit Serum (NRS).

4. The method of claim 3, wherein the NRS reduces the detected level of binding of the detector antibody to the capture antibody in the presence of goat anti-rabbit antibody (GARA) to less than 1.5-times the detected level of the detector antibody binding to the capture antibody in a negative control sample.

5. The method of claim 1, further comprising the steps of
a. selecting a normal rabbit serum based on a serum screening assay comprising the steps of:
i. providing a serum sample,
ii. performing a screening test assay to measure the detected level of binding of a modified rabbit anti-Histoplasma IgG detector antibody comprising the F(ab)′₂ fragment without the F_{c} domain to a rabbit anti-Histoplasma IgG capture antibody in the presence of goat anti-rabbit antibody (GARA) and the serum sample;
iii. performing a control test assay to measure the detected level of binding of the modified rabbit anti-Histoplasma IgG detector antibody comprising the F(ab)′₂ fragment without the F_{c} domain to the rabbit anti-Histoplasma IgG capture antibody in the presence of goat anti-rabbit antibody (GARA) and in the absence of the serum sample;
iv. selecting a serum sample if the binding of the detector antibody to the capture antibody in the presence of the GARA is greater in the screening assay than the control test assay; and
b. combining the detector antibody with the normal rabbit serum selected in step (a) prior to contacting the detector antibody with the bound antigen.

6. The method of claim 1, wherein the step of contacting the antigen binding surface with the analyte is performed in the absence of bovine serum albumin (BSA).

7. The method of claim 1, wherein the antigen binding surface comprises a polyclonal rabbit anti-Histoplasma IgG antibody bound to a microwell plate.

8. The method of claim 1, further comprising the step of contacting the antigen binding surface with a blocking agent prior to contacting the antigen binding surface with the analyte, the blocking agent selected to provide a coefficient of variation for a high positive control of less than 0.2% in 10 or more assay tests.

9. The method of claim 7, wherein the blocking buffer comprises an aqueous solution of a non-animal protein.

10. The method of claim 1, wherein the detected antigen comprises two or more antigens selected from the group consisting of: Histoplasma, Blastomyces, Coccidioides, Paracoccidioides, and Penicillium marneffei endemic mycoses.

11. The method of claim 1, wherein the step of detecting the presence of the detector antibody comprises:
a. contacting the detector antibody comprising biotin and bound to the antigen binding surface with a horseradish peroxidase comprising streptavidin in a manner effective to bind horseradish peroxidase to the detector antibody;
b. contacting the bound horseradish peroxidase with tetramethylbenzidine (TMB) in a manner effective to convert the TMB to a detectable chromophore; and
c. detecting the presence of the detector antibody by measuring the optical density of the chromophore at two or more wavelengths.

12. The method of claim 1, wherein the analyte comprises blood serum or urine.

13. The method of claim 1, further comprising the step of measuring a quantitative calibration curve by performing steps 1a-1d two or more times using an analyte comprising different predetermined quantities of the antigen.

14. The method of claim 13, further comprising the step of providing the antigen concentration in units of antigen mass per unit volume based on correlation to the calibration curve.

15. A method for detecting the presence of a Histoplasma antigen in an analyte, the method comprising the steps of:
a. providing an antigen binding surface comprising a surface-bound anti-Histoplasma capture antibody;
b. contacting the antigen binding surface with a blocking agent selected to provide a coefficient of variation of less than 0.2% in 10 or more assay tests for detection of antigen present in a positive control comprising an antigen;
c. contacting the analyte with the antigen binding surface in a manner effective to bind the antigen to the capture antibody;
d. contacting the bound antigen with a detector antibody in a manner effective to bind the detector antibody to the bound antigen;
e. contacting the bound detector antibody with a chromogenic substrate; and
f. detecting the presence of the chromogenic substrate to detect the presence of the Histoplasma antigen in the analyte.

16. The method of claim 15, wherein the analyte is contacted with the detector antibody in the absence of bovine serum albumin in a manner effective to bind the detector antibody to the bound antigen, and the detector antibody comprises a modified polyclonal rabbit anti-Histoplasma IgG antibody comprising a F(ab)′₂ domain antigen binding domain that is not bound to a F_{c} crystalline domain.

17. The method of claim 15, wherein the step of contacting the antigen binding surface with the analyte is performed in the absence of bovine serum albumin (BSA).

18. The method of claim 15, wherein the analyte and the antigen binding surface are contacted with the detector antibody in the presence of Normal Rabbit Serum (NRS).

19. A method of detecting an antigen comprising the steps of:
a. providing an antigen binding surface;
b. contacting the antigen binding surface with an analyte comprising an antigen in a manner effective bind the antigen to the antigen binding surface;
c. selecting a normal rabbit serum based on a serum screening assay comprising the steps of:
i. providing a serum sample,
ii. performing a screening test assay to measure the detected level of binding of a modified rabbit anti-Histoplasma IgG detector antibody comprising a F(ab)′₂ fragment that is not bound to a F_{c} domain to a rabbit anti-Histoplasma IgG capture antibody in the presence of a goat anti-rabbit antibody (GARA) and the serum sample;
iii. performing a control test assay to measure the detected level of binding of the modified rabbit anti-Histoplasma IgG detector antibody comprising the F(ab)′2 fragment without the Fc domain to the rabbit anti-Histoplasma IgG capture antibody in the presence of the goat anti-rabbit antibody (GARA) and in the absence of the serum sample; and
iv. selecting a screened normal rabbit serum sample when the binding of the detector antibody to the capture antibody in the presence of the GARA is greater in the screening assay than the control test assay; and contacting the bound antigen with a detector antibody;
d. providing a detector antibody adapted to bind to the bound antigen;
e. combining the detector antibody with the screened normal rabbit serum sample selected from the serum screening assay;
f. contacting the detector antibody and the screened normal rabbit serum with the bound antigen in a manner effective to bind the detector antibody to the bound antigen; and
g. detecting the detector antibody bound to the bound antigen.

20. A method of detecting an antigen comprising the steps of:
a. providing an antigen binding surface;
b. contacting the antigen binding surface with an analyte comprising an antigen in a manner effective to bind the antigen to the antigen binding surface;
c. providing a detector antibody adapted to bind to the bound antigen;
d. contacting the detector antibody with the bound antigen in a manner effective to bind the detector antibody to the bound antigen; and
e. detecting the detector antibody bound to the bound antigen;
wherein the method is further described by one or more of criteria selected from the group consisting of:
i. the detector antibody comprises a modified polyclonal rabbit anti-Histoplasma IgG antibody comprising a fragment antigen binding domain that is not bound to a F_{c} crystalline domain,
ii. the step of contacting the antigen bound to the antigen binding surface with the detector antibody is performed in the presence of Normal Rabbit Serum (NRS); and
iii. the step of contacting the antigen binding surface with the analyte is performed in the absence of bovine serum albumin (BSA).

21. The method of claim 20, wherein the detector antibody binds to a bound Histoplasmosa antigen and to one or more bound antigens selected from the group consisting of: Blastomyces, Coccidioides, Paracoccidioiedes and Penicillium mameffei.

22. The method of claim 20, wherein the method is further described by the following criteria:
i. the detector antibody comprises a modified polyclonal rabbit anti-Histoplasma IgG antibody comprising a fragment antigen binding domain that is not bound to a F_{c} crystalline domain,
ii. the step of contacting the antigen bound to the antigen binding surface with the detector antibody is performed in the presence of Normal Rabbit Serum (NRS); and
iii. the step of contacting the antigen binding surface with the analyte is performed in the absence of bovine serum albumin (BSA).

23. A kit for detection of an antigen in an analyte, the kit comprising:
a. a means for capturing an antigen in the analyte to form a bound antigen;
b. a detection antibody composition adapted to bind to the bound antigen; and
c. a means for detecting the detection antibody bound to the antigen.
wherein the kit comprises one or more components selected from the group consisting of:
i. the detector antibody composition comprising a modified polyclonal rabbit anti-Histoplasma IgG antibody comprising a fragment antigen binding domain that is not bound to a F_{c} crystalline domain,
ii. the detector antibody composition further comprising a Normal Rabbit Serum (NRS) that reduces the detected level of binding of the detector antibody to the capture antibody in the presence of goat anti-rabbit antibody (GARA) to less than 1.5-times the detected level of the detector antibody binding to the capture antibody in a negative control sample; and
iii. a blocking agent providing a coefficient of variation of less than 0.2% in 10 or more assay tests for detection of antigen present in a positive control comprising an antigen.

24. The kit of claim 23, further comprising a blocking solution containing the detection antibody and being substantially free of bovine serum albumin.

25. The kit of claim 24, wherein the blocking solution antibody solution further comprises Normal Rabbit Serum.

26. The kit of claim 23, wherein the means for capturing the antigen comprises a microwell plate comprising an anti Histoplasma IgG capture antibody bound to at least one surface of the microwell plate.
